SEQUENCE LISTING (32 sequences)

SEQ ID NO: 1 - protein, 4 aa, Artificial Sequence
  elastin-like peptide (ELP) unit
VPGG

SEQ ID NO: 2 - protein, 4 aa, Artificial Sequence
  elastin-like peptide (ELP) unit
IPGG

SEQ ID NO: 3 - protein, 5 aa, Artificial Sequence
  elastin-like peptide (ELP) unit
VPGXG

SEQ ID NO: 4 - protein, 5 aa, Artificial Sequence
  elastin-like peptide (ELP) unit
AVGVP

SEQ ID NO: 5 - protein, 5 aa, Artificial Sequence
  elastin-like peptide (ELP) unit
IPGXG

SEQ ID NO: 6 - protein, 5 aa, Artificial Sequence
  elastin-like peptide (ELP) unit
IPGVG

SEQ ID NO: 7 - protein, 5 aa, Artificial Sequence
  elastin-like peptide (ELP) unit
LPGXG

SEQ ID NO: 8 - protein, 5 aa, Artificial Sequence
  elastin-like peptide (ELP) unit
LPGVG

SEQ ID NO: 9 - protein, 6 aa, Artificial Sequence
  elastin-like peptide (ELP) unit
VAPGVG

SEQ ID NO: 10 - protein, 8 aa, Artificial Sequence
  elastin-like peptide (ELP) unit
GVGVPGVG

SEQ ID NO: 11 - protein, 9 aa, Artificial Sequence
  elastin-like peptide (ELP) unit
VPGFGVGAG

SEQ ID NO: 12 - protein, 9 aa, Artificial Sequence
  elastin-like peptide (ELP) unit
VPGVGVPGG

SEQ ID NO: 13 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 14 - protein, 691 aa, Artificial Sequence
  proinsulin ELP1-120 fusion protein
FVNQHLCGSHLVEALYLVCGERGFFYTPKTRREAEDLQVGQVELGGGPGAGSLQPLALEGSLQKRGIVEQCCTSICSLYQLENYCNVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 15 - protein, 146 aa, Artificial Sequence
  insulin B chain-insulin A chain-ELP1-20
FVNQHLCGSHLVEALYLVCGERGFFYTPKTGIVEQCCTSICSLYQLENYCNVPGVGVPGVGVPGGGVPGAGVPGVGVPGVGVPGVGVPGGGVPGAGVPGVGVPGVGVPGGGVPGAGVPGVGVPGVGVPGVGVPGGGVPGAGVPGWP

SEQ ID NO: 16 - protein, 181 aa, Artificial Sequence
  proinsulin ELP1-20 fusion protein
FVNQHLCGSHLVEALYLVCGERGFFYTPKTRREAEDLQVGQVELGGGPGAGSLQPLALEGSLQKRGIVEQCCTSICSLYQLENYCNVPGVGVPGVGVPGGGVPGAGVPGVGVPGVGVPGVGVPGGGVPGAGVPGVGVPGVGVPGGGVPGAGVPGVGVPGVGVPGVGVPGGGVPGAGVPGWP

SEQ ID NO: 17 - protein, 136 aa, Artificial Sequence
  proinsulin ELP1-10 fusion protein
FVNQHLCGSHLVEALYLVCGERGFFYTPKTRREAEDLQVGQVELGGGPGAGSLQPLALEGSLQKRGIVEQCCTSICSLYQLENYCNVPGVGVPGVGVPGGGVPGAGVPGVGVPGVGVPGVGVPGGGVPGAGVPGWP

SEQ ID NO: 18 - protein, 206 aa, Artificial Sequence
  proinsulin ELP2-20 fusion protein
FVNQHLCGSHLVEALYLVCGERGFFYTPKTRREAEDLQVGQVELGGGPGAGSLQPLALEGSLQKRGIVEQCCTSICSLYQLENYCNVPGVGVPGVGVPGAGVPGGGVPGAGVPGGGVPGAGVPGGGVPGAGVPGGGVPGAGVPGGGVPGVGVPGAGVPGGGVPGAGVPGGGVPGAGVPGGGVPGAGVPGGGVPGAGVPGGGVPGWP

SEQ ID NO: 19 - protein, 151 aa, Artificial Sequence
  proinsulin ELP1-10 fusion protein
FVNQHLCGSHLVEALYLVCGERGFFYTPKTRREAEDLQVGQVELGGGPGAGSLQPLALEGSLQKRGIVEQCCTSICSLYQLENYCNVPGVGVPGVGVPGAGVPGGGVPGAGVPGGGVPGAGVPGGGVPGAGVPGGGVPGAGVPGGGVPGWP

SEQ ID NO: 20 - protein, 143 aa, Artificial Sequence
  proinsulin ELP1-10(4xGlu) fusion protein
MKRFVNQHLCGSHLVEALYLVCGERGFFYTPKTRREAEDLQVGQVELGGGPGAGSLQPLALEGSLQKRGIVEQCCTSICSLYQLENYCNVPGVGEVPGVGEVPGGGVPGAGVPGVGEVPGVGVPGVGEVPGGGVPGAGVPGWP

SEQ ID NO: 21 - protein, 151 aa, Artificial Sequence
  lispro ELP1-10 fusion protein
FVNQHLCGSHLVEALYLVCGERGFFYTKPTRREAEDLQVGQVELGGGPGAGSLQPLALEGSLQKRGIVEQCCTSICSLYQLENYCNVPGVGVPGVGVPGAGVPGGGVPGAGVPGGGVPGAGVPGGGVPGAGVPGGGVPGAGVPGGGVPGWP

SEQ ID NO: 22 - protein, 151 aa, Artificial Sequence
  aspart ELP1-10 fusion protein
FVNQHLCGSHLVEALYLVCGERGFFYTDKTRREAEDLQVGQVELGGGPGAGSLQPLALEGSLQKRGIVEQCCTSICSLYQLENYCNVPGVGVPGVGVPGAGVPGGGVPGAGVPGGGVPGAGVPGGGVPGAGVPGGGVPGAGVPGGGVPGWP

SEQ ID NO: 23 - protein, 743 aa, Artificial Sequence
  proinsulin ELP2-10 ELP1-120 breakaway fusion protein
FVNQHLCGSHLVEALYLVCGERGFFYTPKTRREAEDLQVGQVELGGGPGAGSLQPLALEGSLQKRGIVEQCCTSICSLYQLENYCNVPGAGVPGGGVPGAGVPGGGVPGAGVPGGGVPGAGVPGGGVPGAGVPGGGRRVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 24 - protein, 21 aa, Homo sapies
GIVEQCCASVCSLYQLENYCN

SEQ ID NO: 25 - protein, 30 aa, Homo sapies
FVNQHLCGSHLVEALYLVCGERGFFYTPKA

SEQ ID NO: 26 - protein, 12 aa, Artificial Sequence
  peptide linker sequence
KDDNPNLPRLVR

SEQ ID NO: 27 - protein, 12 aa, Artificial Sequence
  peptide linker sequence
GAGSSSRRAPQT

SEQ ID NO: 28 - protein, 450 aa, Artificial Sequence
  Elastin-like-peptide (ELP) protein polymer
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 29 - protein, 600 aa, Artificial Sequence
  Elastin-like-peptide (ELP) protein polymer
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 30 - protein, 30 aa, Artificial Sequence
  GLP-1 Gly2 peptide
HGEGTFTSDVSSYLEGQAAKEFIAWLVKGR

SEQ ID NO: 31 - protein, 38 aa, Artificial Sequence
  exendin peptide
HGEGTFTSDLSKQMEEEAVRLFEWLKNGGPSSGAPPPS

SEQ ID NO: 32 - protein, 636 aa, Artificial Sequence
  PB1023 GLP-1 receptor agonist which includes an ELP1-120 fusion
HGEGTFTSDVSSYLEGQAAKEFIAWLVKGRGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

(19) United States
(12) Patent Application Publication
(10) Pub. No.: US 2015/0361154 A1
(43) Pub. Date: Dec. 17, 2015

(54) THERAPEUTIC AGENTS, COMPOSITIONS, AND METHODS FOR GLYCEMIC CONTROL

(71) Applicant: PHASEBIO PHARMACEUTICALS, INC., Malvern,, PA (US)

(72) Inventors: James JOWETT, Malvern, PA (US); Christopher WOODS, Malvern, PA (US); Susan ARNOLD, Malvern, PA (US)

(21) Appl. No.: 14/761,255

(22) PCT Filed: Jan. 15, 2014
(86) PCT No.: PCT/US14/11620

§ 371 Date: Jul. 15, 2015

Related U.S. Application Data
(60) Provisional application No. 61/752,542, filed on Jan. 15, 2013.
(60) Provisional application No. 61/829,074, filed on May 30, 2013.

(51) Int. Cl. C07K 14/62; A61K 38/26; A61K 38/28

(52) U.S. Cl. CPC C07K 14/62; A61K 38/28; A61K 38/26

(57) ABSTRACT

The present invention relates in part to insulin proteins and pharmaceutical compositions having therapeutic advantages.

### PRIORITY

This application claims the benefit of, and claims priority to, U.S. Provisional Application No. 61/752,542 filed Jan. 15, 2013, and U.S. Provisional Application No. 61/829,074 filed May 30, 2013, and the contents of each are hereby incorporated by reference in their entireties.

### FIELD OF THE INVENTION

The present invention relates in part to insulin fusion proteins, and pharmaceutical compositions and combination therapies comprising the same, which provide therapeutic advantages for glycemic control.

### BACKGROUND

The effectiveness of peptide and small molecule drugs is often limited by the half-life of such drugs in the circulation, as well as difficulties in obtaining substantially constant plasma levels. For example, the incretin GLP-1 must be administered at relatively high doses to counter its short half-life in the circulation, and these high doses are associated with nausea, among other things. Murphy and Bloom, Nonpeptidic glucagon-like peptide 1 receptor agonists: A magic bullet for diabetes? PNAS 104 (3):689-690 (2007). Further, the peptide agent vasoactive intestinal peptide (VIP) exhibits a half-life, in some estimates, of less than one minute, making this agent impractical for pharmaceutical use. Domschke et al., Vasoactive intestinal peptide in man: pharmacokinetics, metabolic and circulatory effects, Gut 19:1049-1053 (1978); Henning and Sawmiller, Vasoactive intestinal peptide: cardiovascular effects, Cardiovascular Research 49:27-37 (2001). A short plasma half life for peptide drugs is often due to fast renal clearance as well as to enzymatic degradation during systemic circulation.

Insulin, or derivatives thereof, suffer from similar difficulties, but also present additional challenges. The islet cells of the pancreas secrete a basal level of insulin between meals and overnight to ensure that the blood glucose level remains within an appropriate range, particularly to prevent nocturnal hypoglycemia. Following ingestion of a meal, large quantities of insulin are released into the circulation in preparation for the influx of glucose into the circulation following digestion and absorption. To optimize control of blood sugar levels and prevent hyperglycemia, this so-called prandial insulin must be released very rapidly; in normal individuals the peak level of insulin occurs 30-45 minutes after commencement of a meal.

Current insulin therapies for treatment of type 1 and type 2 diabetes essentially take two forms: basal insulin to provide a low level of insulin throughout the day and insulin for injection immediately prior to a meal to mimic the insulin normally released by the body at meal times. The challenge for basal insulin products is to provide a constant level of insulin, without a large difference between peak and trough levels, ideally with injection no more than once a day, preferably once a week. For prandial insulin, the molecule needs to be absorbed from the subcutaneous injection site very rapidly, with the levels decaying to near basal within two hours or so.

Currently available prandial insulin formulations have high insulin concentration, and these insulin molecules self-assemble into hexamers in the presence of zinc. Thus, the insulin molecules generally exist in solution as a dynamic equilibrium of hexamers, dimers, and monomers, the proportions of which depend on the concentration and pH of the solution. In commercial insulin formulations at neutral pH, this equilibrium strongly favors the zinc-stabilized hexamer, which is advantageous for shelf life stability as monomeric insulin is generally unstable and subject to deamidation. On subcutaneous injection, hexameric insulin is poorly absorbed and must dissociate to dimer and monomer for uptake into the circulation, leading to a significant delay in reaching maximal levels.

Rapid-acting insulin's have an altered amino acid sequence to de-stabilize the dimer and hexamer so that the insulin is converted to the monomer form more rapidly. But the currently marketed products are still significantly slower to reach peak levels than endogenously released insulin, necessitating injection 20 minutes before a meal for optimal control. A number of approaches are currently under investigation to further accelerate uptake of injected insulin, including warming the site of injection to increase blood flow, injection of an enzyme to breakdown the hyaluranan layer of the subcutaneous adipose layer (reviewed by Heinemann and Muchmore, 2012. Ultrafast-Acting Insulins: State of the Art Journal of Diabetes Science and Technology 6, 728-742). There is increasing interest in extending the use of pumps for delivery of insulin by incorporating data from continuous glucose monitoring, ultimately leading to a closed-loop system requiring little or no input from the user. However, the current rapid acting insulins are too slow to really enable such a feedback loop system as their effect on the blood glucose levels is significantly delayed. A more rapid-acting insulin that is stable in a physiologically acceptable solution in a pump reservoir would enable to development of such so-called “artificial pancreas” systems (Cenzig, 2012. Undeniable Need for Ultrafast-Acting Insulin: The Pediatric Perspective Journal of Diabetes Science and Technology 6, 797-801).

The converse situation pertains to development of a basal insulin product, where the aim is to have sustained uptake from the injection site, or an insulin molecule with a protracted circulatory half-life, or a combination of the two. The most popular basal insulin currently on the market is insulin glargine, but this product does not provide a full 24 hours of coverage.

Thus, there remains a need for insulin therapies that display improved pharmaceutical properties, both in terms of, for example, improved basal insulin products and more rapid acting prandial insulins. In particular, there is a need for basal and rapid-acting insulins that are compatible and can be formulated together to enable administration once a day, providing the basal component plus prandial coverage for the meal that would otherwise lead to the highest or most prolonged glucose excursion, which is usually breakfast.

Other objects of the invention will be apparent from the following description of the invention.

### SUMMARY OF THE INVENTION

The present invention provides insulin-based pharmaceutical agents, and compositions, combinations, and formulations thereof. The agents display one or more advantages selected from: sustained release, rapid action (due to, for example, loss of or reduced hexamer formation), and reduced activation of the IGF receptor. The present invention relates in various aspects to fusion partners for insulin that provide sustained release (e.g., long-acting) or rapid-action profiles, which can be administered individually, or administered as co-therapy either separately or together.

In one aspect, the present invention provides a fusion protein comprising an insulin B chain and insulin A chain, and a fusion partner of from 5 to 200 amino acids, such as from about 50 to about 150 amino acid residues, the fusion partner inhibiting insulin multimer (e.g., hexamer) formation. In some embodiments, the fusion partner has an extended conformation, which may form a random coil or conformation of repeating beta-turns, which may be the result of a pattern of proline residues and/or overall amino acid composition of the fusion partner's primary sequence. In some embodiments, the sequence of the fusion partner contains less than about 35%, or less than about 30%, or less than about 25%, or less than about 20% hydrophobic residues, excluding alanine, glycine, and proline as hydrophobic residues. For example, hydophobic residues in this context include leucine, isoleucine, valine, methionine, cysteine, tryptophan, phenylalanine, tyrosine, and histidine. The fusion partner may include one or more positively charged residues to reduce hydrophobicity of the fusion partner. In some embodiments, the sequence that reduces or eliminates hexamer formation does not induce sustained release from an injection site. In further embodiments, the sequence stabilizes the insulin in monomeric or substantially monomeric form in a physiologically compatible solution. In these aspects, the invention provides therapeutic agents and pharmaceutical compositions or formulations for providing prandial insulin.

In another aspect, the invention provides pharmaceutical compositions and formulations for providing sustained glycemic control comprising an effective amount of a fusion protein, the fusion protein comprising an insulin amino acid sequence (e.g., an insulin A chain and an insulin B chain) and a fusion partner having an amino acid sequence that provides a sustained release from an injection site, and pharmaceutical excipients to achieve the sustained release. In some embodiments, the fusion partner has an extended conformation, which may form a random coil or conformation of repeating beta-turns, which may be the result of a pattern of proline residues and/or overall amino composition of the fusion partner. The fusion partner in these embodiments may exhibit a phase transition at around 35° C., or around peripheral body temperature, so as to form a matrix or drug depot that results in a sustained release of the drug from an injection site. These aspects are useful for providing basal insulin in diabetic patients, for example.

In some embodiments, the pharmaceutical agents and compositions described herein provide the benefit of having reduced or negligible binding and/or activation at the IGF receptor, so as to make the agent, compositions, and formulations of the invention particularly suitable for chronic therapy of either type I or type II diabetic patients, for example.

In some embodiments, the invention provides co-formulations and combination therapies of the active agents described herein, including co-formulations and combination therapies of basal insulin and rapid-acting insulin fusion proteins, as well as co-formulations of the insulin agents with other agents, such as GLP-1 receptor agonists. For example, in some embodiments, co-therapy or co-formulation of the basal insulin and rapid-acting insulin described herein provides rapid and sustained insulin action for an entire day from a single injection. Further, co-formulation of the sustained release fusion protein with GLP-1 receptor agonist can provide synergistic effects for glycemic control, including in some embodiments providing basal insulin levels with a once weekly administration.

In another aspect, the invention provides methods of treating diabetes, hypoinsulinemea, or hyperglycemia, involving administering a pharmaceutical composition, formulation, or combination therapy described herein. In some embodiments, the patient has type 1 diabetes or type 2 diabetes, or is prediabetic. In some embodiments, the method comprises administering a pharmaceutical composition described herein at a frequency of from about 1 to about 30 times per month, including about once weekly, or about two or three times per week, or about once daily. In some embodiments, the method comprises administering the pharmaceutical composition subcutaneously or intramuscularly. In some embodiments, the method provides for long term therapy, where the agent is administered for a plurality of years. In the case of administering the prandial insulin, in some embodiments the method comprises administering the pharmaceutical composition immediately before a meal, for example, about 15 minutes, about 10 minutes, about 5 minutes, or less than 1 minute before commencing a meal. In some embodiments, peak levels of insulin are obtained at from 30-45 minutes after administration and/or after commencement of the meal.

In some embodiments, the method comprises administering one or more pharmaceutical agents or compositions described herein via a pump system with, optionally, a closed loop system to control the amount of insulin delivered based on, for example, the level of glucose in the blood, as determined by a glucose monitoring system. The system may control, and administer, both a basal insulin (e.g., as described herein), and rapid-acting insulin (e.g., as described herein), and may further control administration of a GLP-1 receptor agonist, as also described herein.

### BRIEF DESCRIPTION OF THE FIGURES

FIG. 1A shows the human proinsulin sequence (SEQ ID NO: 13). The proinsulin sequence consists of the B and A chains linked with the C peptide. The C peptide is removed to form mature insulin following enzymatic cleavage at the two adjacent dibasic sites (underlined in italics).

FIG. 1B shows a diagram of a construction termed PE0139 or INSUMERA or Insulin-ELP1-120 having 120 ELP units fused to the C-terminus of the insulin A chain.

FIG. 2 shows a map of the pPE0139 plasmid.

FIG. 3 shows the amino acid sequence of a proinsulin ELP1-120 fusion protein (SEQ ID NO: 14). The proinsulin sequence (underlined) is fused to the ELP1-120 sequence. The amino acid sequence optionally includes an initiation methionine residue at the N terminus.

FIG. 4 shows a non-reducing SDS-PAGE experiment. Non-reducing SDS-PAGE showed the expected decreased fusion protein molecular weight following enzymatic processing as the C-peptide was cleaved. Lane 1: SEEBLUE® Plus2 pre-stained standard (INVITROGEN), lane 2: ELP1-120, lane 3: Proinsulin ELP1-120, lane 4: Insulin ELP-120 3 μg, lane 5: Insulin ELP1-120 6 μg, lane 6: SEEBLUE® Plus2 pre-stained standard (INVITROGEN).

FIG. 5 shows an anti-insulin B chain western blot. An anti-insulin B chain western blot was performed to confirm presence of both A and B chains fused to ELP. The data showed presence of B-chain under non-reducing conditions indicating disulfide bond formation between the A and B chains. Reduction of the fusion protein and disulfide bonds resulted in removal of B chain from the fusion. Lane 1: reduced Insulin ELP fusion showing absence of B chain, lane 2: Non-reduced Insulin ELP fusion showing presence of B-chain, lane 3: ELP1-120, lane 4: Proinsulin ELP fusion showing presence of B-chain.

FIG. 6 shows ESI-MS data on unprocessed insulin-ELP1-120. Electrospray ionization mass spectrometry confirmed the mass of unprocessed Proinsulin ELP fusion of 57008.5 Da (SGS Mscan Codes 104531 & 104532). Additional salt adducts were present.

FIG. 7 shows ESI-MS data on processed pPE0139. Electrospray ionization mass spectrometry confirmed the mass of mature Insulin ELP fusion following enzymatic removal of the C-peptide (SGS M-scan Codes 107610). ESI-MS of Insulin ELP showed a main product peak with a molecular mass of approximately 53298 Da indicating mature Insulin ELP following C-peptide cleavage. Minor peaks are likely attributable as partially degraded fusion or salt adducts.

FIG. 8 shows blood glucose lowering in normal mice with Insulin-ELP1-120 fusion as compared to insulin glargine.

FIG. 9 shows INSUMERA (PE0139) dosing in a diabetes mellitus type I (type 1 diabetes, T1DM) mouse model. Specifically, single dose data is shown. The results demonstrate greater duration of glucose lowering for INSUMERA, as compared to equimolar LANTUS (insulin glargine, SANOFI-AVENTIS) dosing. STZ is streptozotocin; the untreated group refers to normal, non-diabetic animals; N=8 per group.

FIG. 10 shows INSUMERA (PE0139) dosing in a diabetes mellitus type 1 (type 1 diabetes, T1DM) mouse model. Specifically, daily dosing data is shown. The results demonstrate the superiority of INSUMERA, as compared to LANTUS (insulin glargine, SANOFI-AVENTIS), with regards to activity and half-life. STZ is streptozotocin; the untreated group refers to normal, non-diabetic animals; at the 6 h time point, N=5 for the 25 mg and 50 mg/kg groups; at the 8 h time point, N=3 for the 25 mg/kg group and n=2 for the 50 mg/kg group; at the 24 h time point, N=1 for the 25 mg/kg and N=7 for the 5 mg/kg groups.

FIG. 11A shows INSUMERA (PE0139) low dose titration in a diabetes mellitus type 1 (type 1 diabetes, T1DM) mouse model as compared to LANTUS (insulin glargine, SANOFI-AVENTIS). Specifically, FIG. 11A shows a single s.c. dose. STZ is streptozotocin; the untreated group refers to normal, non-diabetic animals; N=8 for LANTUS, PE0139 1 mg/kg and untreated groups; N=7 for the PE0139 3.33 mg/kg group.

FIG. 11B shows INSUMERA (PE0139) low dose titration in a diabetes mellitus type I (type 1 diabetes, T1DM) mouse model as compared to LANTUS (insulin glargine, SANOFI-AVENTIS). Specifically, FIG. 11B shows 14 days of daily s.c. dosing. STZ is streptozotocin; the untreated group refers to normal, non-diabetic animals; N=8 for LANTUS, PE0139 1 mg/kg and untreated groups; N=7 for the PE0139 3.33 mg/kg group.

FIG. 12A shows that INSUMERA (PE0139) has significantly increased glycemic control relative to LANTUS (insulin glargine, SANOFI-AVENTIS). A reduction of 27-39% is seen in area under the curve (AUC) blood glucose on days 1, 3, 7 and 14 relative to Lantus. Specifically, FIG. 12A shows day 1 of compound administration and the blood glucose AUC at 0-24 hrs.

FIG. 12B shows that INSUMERA (PE0139) has significantly increased glycemic control relative to LANTUS (insulin glargine, SANOFI-AVENTIS). A reduction of 27-39% is seen in area under the curve (AUC) blood glucose on days 1, 3, 7 and 14 relative to Lantus. Specifically, FIG. 12B shows day 14 of compound administration and the blood glucose AUC at 0-24 hrs.

FIG. 13A shows that INSUMERA (PE0139) achieves a long half-life with a small peak to trough ratio following a subcutaneous injection. Specifically, FIG. 13A shows pharmacokinetic (PK) drug levels following a single s.c. injection in diabetic swine.

FIG. 13B shows that INSUMERA (PE0139) achieves steady state peak to trough pharmacokinetic (PK) levels following daily subcutaneous injections. Specifically, FIG. 13B shows daily s.c. injections in diabetic swine for 2 weeks; PK levels measured prior to dosing.

FIG. 14 shows the design of an exemplary fast acting insulin in accordance with the disclosure. The sequence of insulin B chain-insulin A chain-ELP1-20 is shown (SEQ ID NO:15).

FIG. 15 shows production of the fast acting insulin and ion exchange chromatography.

FIG. 16 shows potency of the fast acting insulin as compared to INSUMERA, and shows that the potencies are comparable.

FIG. 17 shows that plasmid pPE0224 encodes the proinsulin ELP1-20 fusion protein.

FIG. 18 shows the amino acid sequence of a proinsulin ELP1-20 fusion protein (SEQ ID NO:16).

FIG. 19 shows that plasmid pPE0262 encodes the proinsulin ELP1-10 fusion protein.

FIG. 20 shows the amino acid sequence of proinsulin ELP1-10 fusion protein (SEQ ID NO:17).

FIG. 21 shows the plasmid pPE0259 encodes the proinsulin ELP2-20 fusion protein.

FIG. 22 shows the amino acid sequence of proinsulin ELP2-20 fusion protein (SEQ ID NO:18).

FIG. 23 shows that plasmid pPE0266 encodes the proinsulin ELP2-10 fusion protein.

FIG. 24 shows the amino acid sequence of proinsulin ELP1-10 fusion protein (SEQ ID NO:19).

FIG. 25 shows that plasmid pPE0283 encodes the proinsulin ELP1-10(4xGlu) fusion protein.

FIG. 26 shows the amino acid sequence of proinsulin ELP1-10(4xGlu) fusion protein (SEQ ID NO:20).

FIG. 27 shows that plasmid pPE0284 encodes the lispro ELP1-10 fusion protein.

FIG. 28 shows the amino acid sequence of lispro ELP1-10 fusion protein (SEQ ID NO:21).

FIG. 29 shows that plasmid pPE0285 encodes the lispro ELP1-10 fusion protein.

FIG. 30 shows the amino acid sequence of aspart ELP1-10 fusion protein (SEQ ID NO:22).

FIG. 31 shows that plasmid pPE0289 encodes the ELP2-10 ELP1-120 breakaway fusion protein.

FIG. 32 shows that plasmid pPE0285 encodes the lispro ELP1-10 fusion protein.

FIG. 33 shows the amino acid sequence of proinsulin ELP2-10 ELP1-120 breakaway fusion protein (SEQ ID NO:23). The underlined sequence is proinsulin, the sequence in bold is ELP2-10, and the sequence in italics is ELP1-120. The two underlined arginines are the trypsin site freeing the two constructs.

FIG. 34 shows the effect of PE0244 and PE0139, and the combination, on blood glucose levels in STZ mice.

FIG. 35A shows that a 3 μM, INS-ELP(B30) and PE0139 exhibit binding to the insulin receptor, as shown with a Biacore optical biosensor.

FIG. 35B shows that a 3 μM, INS-ELP(B30) and PE0139 do not show detectable binding to the IGF-1 receptor using a Biacore optical biosensor.

FIG. 36A and FIG. 36B show that INS-ELP(B30) and PE0139 show similar binding to the insulin receptor.

FIG. 37 shows that dose escalation of PE0139 in db/db mice exhibits increasing effect on fasting and post-prandial glucose.

FIG. 38 shows that the addition of a fixed dose of PB1023 (GLP-1-ELP1-120) to PE0139 further enhances post-prandial control in db/db mice.

FIG. 39 shows that PB1023 dose escalation shows increasing effect on fasting and post-prandial glucose in db/db mice.

FIG. 40 shows that the addition of fixed dose of PE0139 to PB1023 enhances fasting and post-prandial control in db/db mice.

FIG. 41 shows that PE0139 dose escalation shows little effect on blood glucose, and that PB1023 dose escalation shows increasing effect on fasting glucose, and limited impact on post-prandial glucose.

FIG. 42 shows that combination of low doses of PE0139 and PB1023 have enhanced fasting and post-prandial control.

### DETAILED DESCRIPTION

The present invention provides insulin-based pharmaceutical agents, compositions, formulations, combinations, and co-therapies that exhibit therapeutic advantages, such as sustained glycemic control, rapid action (due to, for example, loss of or reduced insulin hexamer formation), and/or reduced or negligible binding and/or activation of the IGF Receptor. Also provided are methods and uses for treating disease, including hyperglycemia, hypoinsulinemia, diabetes (including type 1 and type 2), metabolic disease, and obesity, with the agents, compositions, and formulations of the present invention.

In one aspect, the present invention provides a fusion protein comprising an insulin B chain and insulin A chain, and a fusion partner of from 5 to 200 amino acids, such as from about 50 to about 150 amino acids, the fusion partner inhibiting insulin multimer (e.g., hexamer) formation. In some embodiments, the fusion partner has an extended conformation, which may form a random coil or conformation of repeating beta-turns, which may result from a pattern of proline residues or overall amino acid composition of the fusion partner's primary sequence. In some embodiments, the sequence of the fusion partner contains less than about 35%, less than about 30%, or less than about 25%, or less than about 20% hydrophobic residues (excluding alanine, glycine, and proline as hydrophobic residues). For example, hydophobic residues in this context include leucine, isoleucine, valine, methionine, cysteine, histidine, phenylalanine, tyrosine, and tryptophan. In some embodiments, the sequence that reduces or eliminates hexamer formation does not induce sustained release from an injection site, or exhibit a phase transition at body temperature. In further embodiments, the sequence stabilizes the insulin in monomeric or substantially monomeric form in a physiologically compatible solution. In these aspects, the invention provides therapeutic agents and pharmaceutical compositions for providing prandial insulin, based on the rapid onset of action described herein. In some embodiments, the rapid acting insulin reaches peak action within about 45 minutes or less, or within about 35 minutes or less, or within about 30 minutes or less.

In other embodiments, the present invention provides a fusion protein comprising an insulin B chain and insulin A chain, and a fusion partner of from about 400 to about 1000 amino acids, such as from about 400 to about 800 amino acids, the fusion partner exhibiting a phase transition at body temperature so as to provide a sustained release of the active agent from the injection site. In some embodiments, the fusion partner has an extended conformation, which may form a random coil or conformation of repeating beta-turns, which may result from a pattern of proline residues or overall amino acid composition in the amino acid sequence. In these aspects, the invention provides therapeutic agents and pharmaceutical compositions and formulations for providing basal insulin, based on the sustained release and slow clearance from the circulation as described herein. In some embodiments, the sustained release insulin is dosed for once daily or once weekly administration.

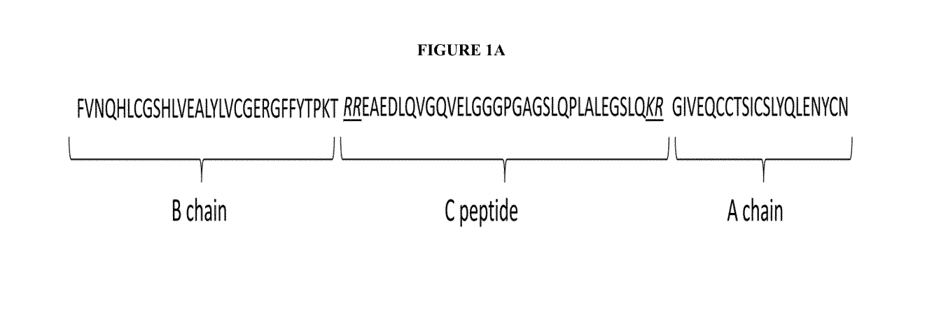

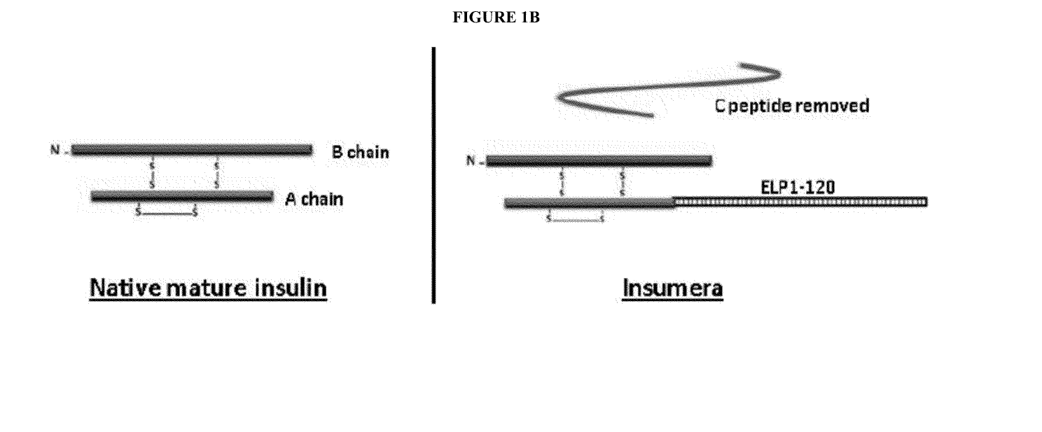

In various embodiments the insulin amino acid sequence comprises an A chain and a B chain amino acid sequence and the A chain and B chain have the amino acid sequence of SEQ ID NO:13 (FIG. 1), optionally having from 1 to 8 or from 1 to 5 amino acid insertions, deletions, or substitutions, collectively. For example, positions 3, 28, 29, and 30 of the insulin B chain can be substituted, as well as position 21 of the A chain, including with K3, G21, K28, D28, P29, Q29. In some embodiments, from one to five amino acids are added to the C-terminus of the B chain, or T30 of the B chain is removed. Such alterations may optionally correspond to the sequences of lispro, aspart, glulisine, glargine and detemir. In some embodiments, the amino acid sequence that provides a slow absorption from the injection site, or which reduces or eliminates insulin multimer formation, is covalently bound to the insulin A chain. In another embodiment, the A chain and B chain are bound by one or more disulfide bonds or attached through a peptide or chemical linker.

In various embodiments, the fusion partner is covalently bound to the insulin A chain, and the fusion partner may comprise elastin-like peptide (ELP) units, for example, from about 5 to about 180 ELP units, depending on whether rapid or sustained biological action are desired. In some embodiments, the ELP comprises repeats of VPGXG (SEQ ID NO: 3), where each X is independently selected from alanine, arginine, asparagine, aspartic acid, glutamic acid, glutamine, glycine, histidine, isoleucine, leucine, lysine, methionine, phenylalanine, serine, threonine, tryptophan, tyrosine and valine residues. In some embodiments, X is independently selected from V, I, L, A, G, and W. In some embodiments, the ELP comprises repeats of AVGVP (SEQ ID NO: 4), IPGVG (SEQ ID NO: 6), or LPGVG (SEQ ID NO: 8). The size and amino acid composition of the fusion partner is selected to provide either rapid action, or sustained activity, as described in more detail herein.

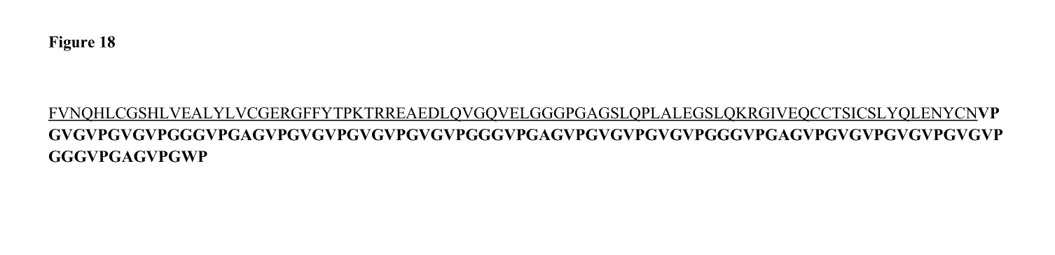

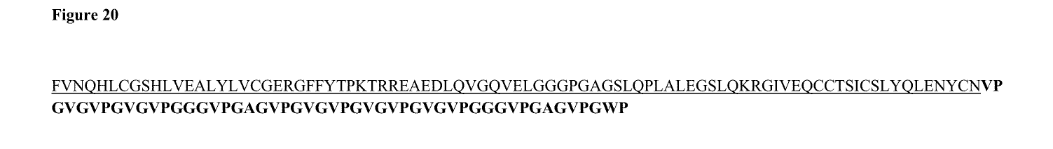

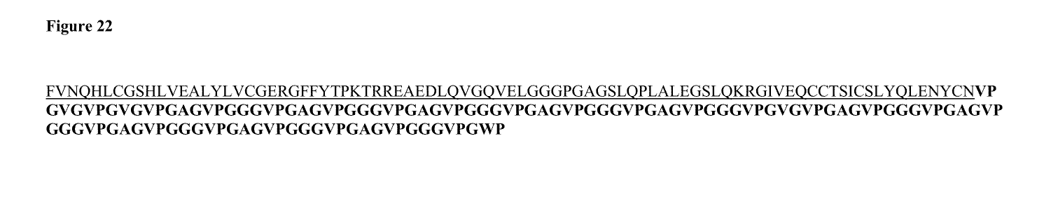

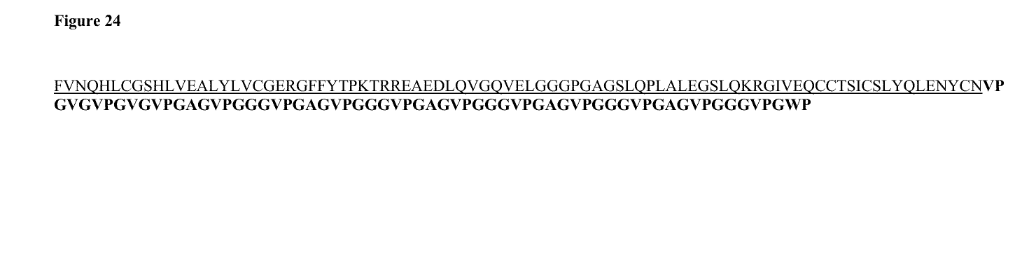

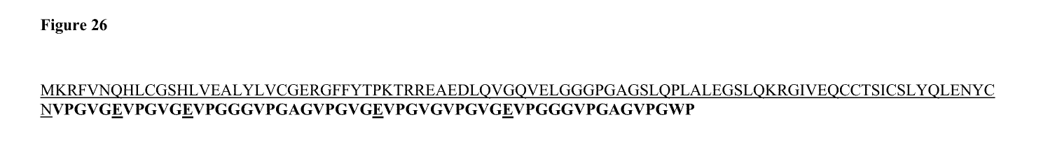

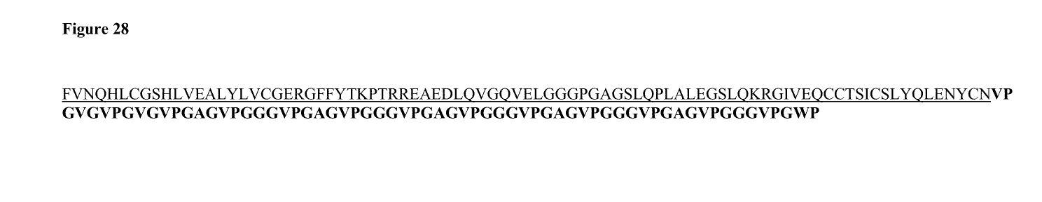

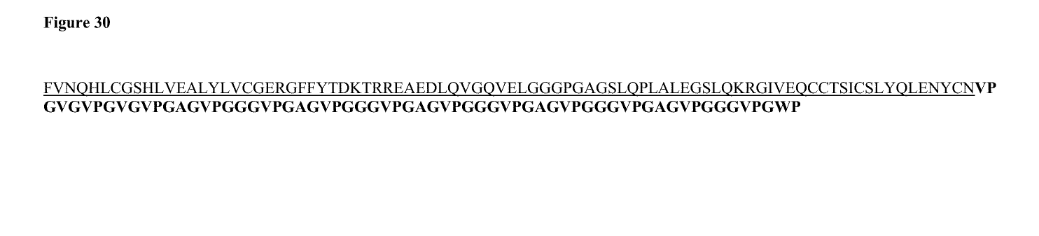

In some embodiments for providing rapid-acting insulin, the fusion may contain from 1 to about 10 negatively charged amino acids such as, for example, glutamic acid and/or aspartic acid to increase the negative charge and reduce hydrophobicity of the fusion partner. In some embodiments, the ELP is comprised of about 10 to about 25 VPGXG units (SEQ ID NO: 3), where X is independently selected from V, G, and A where G and A are present in an approximately equivalent amount as guest residues (e.g., within about 1 or 2), and V is present at from about 2 to about 3 times more than G or A. Alternatively, the ELP units are AVGVP (SEQ ID NO:4). For example, an exemplary fusion or fusion partner for rapid action insulin is exemplified by SEQ ID NO:16 (FIG. 18) or SEQ ID NO:17 (FIG. 20). In other embodiments, the ELP is comprised of about 10 to about 25 VPGXG units (SEQ ID NO: 3), where X is independently selected from V, G, and A where G and A are present in an approximately equivalent amount as the guest residue (e.g., within about 1 or about 2), and V is present at from about 2 to about 3 times less than G or A. For example, an exemplary fusion protein or fusion partner for rapid action insulin is exemplified by SEQ ID NO:18 (FIG. 22) or SEQ ID NO:19 (FIG. 24) or SEQ ID NO:21 (FIG. 28) or SEQ ID NO:22 (FIG. 30). In other embodiments, the ELP is comprised of about 10 to about 25 VPGXG units (SEQ ID NO: 3) as above, where positively charged residues are inserted between a portion of the ELP units, for example, in from about 2 to about 6 locations (e.g., about 3, 4, or 5 locations). An exemplary fusion proteins or fusion partners according to these embodiments is shown in FIG. 26 (SEQ ID NO:20). Alternatively, positively charges residues may be inserted as one or more guest residues of the ELP sequence, or at another position of the ELP unit.

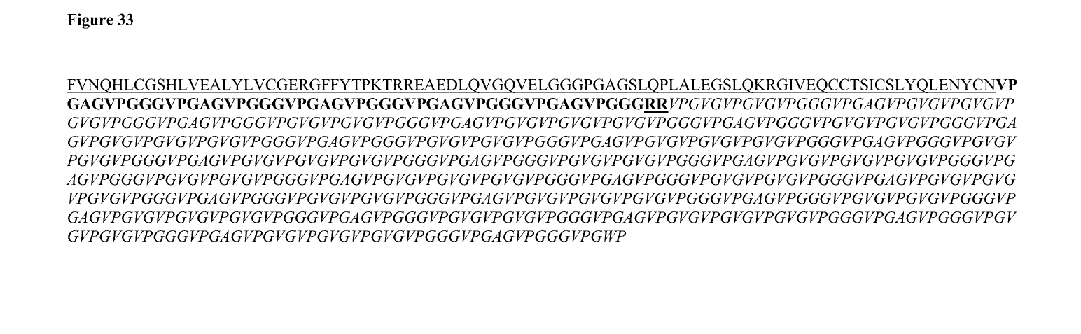

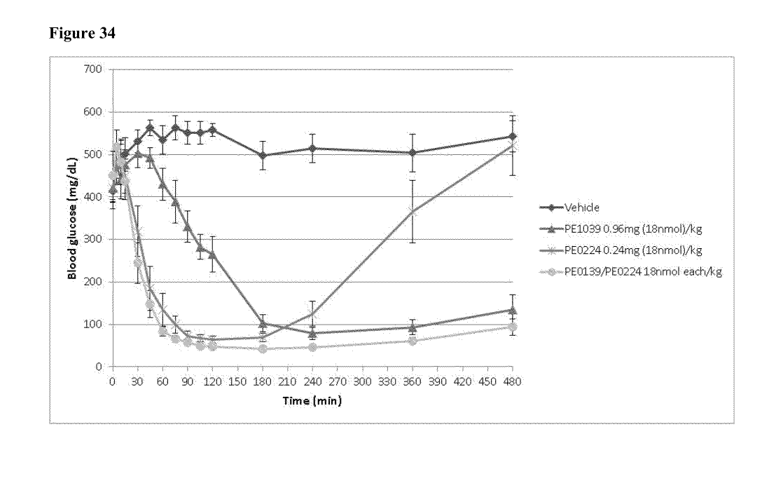

In some embodiments, the fusion protein providing for sustained release and rapid action are produced together as a “breakaway” fusion, and released during processing by the action of protease (e.g., Trypsin). Such a design is shown in FIG. 33 (SEQ ID NO:23). In such embodiments, a 1:1 co-formulation (or other defined molar ratio) of rapid-action and sustained action insulins can be conveniently prepared. In various embodiments, the breakaway construct comprises from one to five rapid acting insulin units (e.g., one or two), with from one to five (e.g., one or two) sustained release insulin units, each being connected by a protease cleavage site.

In some aspects, the invention provides the sustained release fusion protein as a pharmaceutical composition with excipients that allow for sustained release, and/or keeps the rapid action version in a largely monomeric state. For example, the pharmaceutical composition or formulation may have the ionic strength of about 110 mM sodium chloride with about 20 mM histidine. In some embodiments, the pharmaceutical composition is formulated for administration about once per week, or once daily.

In some aspects, the invention provides a use and method of treating diabetes or hypoinsulinemea, comprising administering an effective amount of the pharmaceutical composition or formulation described herein to a patient in need thereof. In some embodiments, the patient has type 1 or type 2 diabetes or is prediabetic, and/or has metabolic disease or clinical obesity. In some embodiments, the pharmaceutical composition for sustained release is administered at a frequency of from 1 to about 30 times per month, or about once weekly, or about 2 to 3 times per week, or about once daily. In some embodiments, the method also delivers prandial insulin. For example, the method may comprise administering the pharmaceutical composition comprising the rapid acting insulin immediately before a meal to provide prandial insulin, for example, about 15 minutes, about 10 minutes, about 5 minutes, or less than 1 minute before a meal. The invention further provides methods and uses of insulin fusion proteins described herein for use in the manufacture of medicaments for treatment of such diseases.

In some embodiments, the method comprises administering the pharmaceutical composition subcutaneously or intramuscularly. In some embodiments, the agents and compositions described herein are suitable for chronic therapy, due to reduced activation of the IGF Receptor. In some embodiments, the method provides for long term therapy, where the agent is administered for a plurality of years, including more than about 2 years, more than about 5 years, more than about 10 years, or more.

In some embodiments, the methods comprise administering the pharmaceutical composition via a pump system with, optionally, a closed loop system to control the amount of insulin delivered based on the level of glucose in the blood, as determined by a glucose monitoring system. The pump system may deliver both rapid action and sustained action insulin, based on, for example, regular frequency of administration for sustained action insulin, with rapid action insulin administered in response to rising glucose levels or timed for delivery prior to commencing a meal.

In various embodiments, the amino acid sequence providing for the therapeutic advantage (e.g., sustained release or rapid action) has an extended conformation, or random coil, as a result of a pattern of proline residues in the fusion partner. The pattern may be a repeating pattern of 13 turns. For example, the amino acid sequence in some embodiments is an elastin-like peptide (ELP) amino acid sequence. In some embodiments, the ELP comprises repeats of VPGXG (SEQ ID NO: 3), where each X is independently selected from alanine, arginine, asparagine, aspartic acid, glutamic acid, glutamine, glycine, histidine, isoleucine, leucine, lysine, methionine, phenylalanine, serine, threonine, tryptophan, tyrosine and valine residues. In some embodiments, the ELP amino acid sequence comprises repeats of AVGVP (SEQ ID NO: 4), IPGVG (SEQ ID NO: 6), or LPGVG (SEQ ID NO: 8). In various embodiments, the ELP comprises at least 5, or at least 10, or at least 15, or at least 30, or at least 60, or at least 90, or at least 120, or at least 180 repeats of an ELP amino acid unit (as described herein). In some embodiments, the ELP is less than about 60 ELP units, or less than about 30 ELP units, to avoid formation of insulin multimers. In some embodiments, the fusion partner has from about 5 to about 30 ELP units, such as from about 10 to about 25, including about 10, about 15, about 20, and about 25. In these embodiments, the ELP does not induce a sustained release from the injection site, and does not exhibit a reverse phase transition at body temperature. In another embodiment, the ELP amino acid sequence includes at least 60 ELP units, including at least 90 ELP units, and including about 120 ELP units, and has a transition temperature of just less than 37° C. in normal saline, to thereby provide the sustained release action. For example, in some embodiments, the transition temperature of the ELP can be about 36° C., or about 35° C., or about 34° C., or about 33° C., or about 32° C., or about 31° C., or about 30° C.

In some embodiments, the amino acid sequence providing therapeutic advantages forms a random coil or non-globular extended structure or unstructured biopolymer, including a biopolymer where at least 50% of the amino acids are devoid of secondary structure as determined by the Chou-Fasman algorithm. In such embodiments, the fusion partner might be described as a random coil conformation. In yet another embodiment, the amino acid sequence providing a sustained release is a protein having an extended, non-globular structure, or a random coil structure, as described in more detail below, which provides, among other things, a large hydrodynamic radius to reduce the rate of elimination from circulation.

In various embodiments, the invention provides co-formulations of basal insulin (including sustained release fusion proteins described herein) and rapid action insulin (which may include fusion proteins described herein), where basal and prandial insulin can be administered once or twice a day in a single injection (e.g., prior to breakfast). In these or other embodiments, the co-formulations include one or more GLP-1 receptor agonists, which may provide for superior glycemic control, or control of underlying or complicating metabolic disease or excessive weight (e.g., clinical obesity).

Further aspects and embodiments of the invention will be apparent from the following detailed descriptions.

### Insulin Amino Acid Sequences

Insulin injections, e.g. of human insulin, can be used to treat diabetes. The insulin-making cells of the body are called β-cells, and they are found in the pancreas gland. These cells clump together to form the “islets of Langerhans,” named for the German medical student who described them.

The synthesis of insulin begins at the translation of the insulin gene, which resides on chromosome 11. During translation, two introns are spliced out of the mRNA product, which encodes a protein of 110 amino acids in length. This primary translation product is called preproinsulin and is inactive. It contains a signal peptide of 24 amino acids in length, which is required for the protein to cross the cell membrane. Human proinsulin consists of A and B chains linked together with the 31 amino acid C peptide (FIG. 1).

Once the preproinsulin reaches the endoplasmic reticulum, a protease cleaves off the signal peptide to create proinsulin. Specifically, once disulfide bonds are formed between the A and B chains the proinsulin is converted into mature insulin in vivo by removal of the C peptide by a trypsin/carboxypeptidase B-like system. Proinsulin consists of three domains: an amino-terminal B chain, a carboxyl-terminal A chain, and a connecting peptide in the middle known as the C-peptide. Insulin is composed of two chains of amino acids named chain A (21 amino acids—GIVEQCCASVCSLYQLENYCN) (SEQ ID NO:24) and chain B (30 amino acids FVNQHLCGSHLVEALYLVCGERGFFYTPKA) (SEQ ID NO:25) that are linked together by two disulfide bridges. There is a 3rd disulfide bridge within the A chain that links the 6th and 11th residues of the A chain together. In most species, the length and amino acid compositions of chains A and B are similar, and the positions of the three disulfide bonds are highly conserved. For this reason, pig insulin can replace deficient human insulin levels in diabetes patients. Today, porcine insulin has largely been replaced by the mass production of human proinsulin by bacteria (recombinant insulin).

Insulin molecules have a tendency to form dimers in solution, and in the presence of zinc ions, insulin dimers associate into hexamers. Whereas monomers of insulin readily diffuse through the blood and have a rapid effect, hexamers diffuse slowly and have a delayed onset of action. In the design of recombinant insulin, the structure of insulin can be modified in a way that reduces the tendency of the insulin molecule to form dimers and hexamers but that does not interrupt binding to the insulin receptor. In this way, a range of preparations are made, varying from short acting to long acting.

Within the endoplasmic reticulum, proinsulin is exposed to several specific peptidases that remove the C-peptide and generate the mature and active form of insulin. In the Golgi apparatus, insulin and free C-peptide are packaged into secretory granules, which accumulate in the cytoplasm of the β-cells. Exocytosis of the granules is triggered by the entry of glucose into the beta cells. The secretion of insulin has a broad impact on metabolism.

There are two phases of insulin release in response to a rise in glucose. The first is an immediate release of insulin. This is attributable to the release of preformed insulin, which is stored in secretory granules. After a short delay, there is a second, more prolonged release of newly synthesized insulin.

Once released, insulin is active for only a brief time before it is degraded by enzymes. Insulinase found in the liver and kidneys breaks down insulin circulating in the plasma, and as a result, insulin has a half-life of only about 6 minutes. This short duration of action results in rapid changes in the circulating levels of insulin.

Insulin analogs have been developed with improved therapeutic properties (Owens et al., 2001, Lancet 358: 739-46; Vajo et al., 2001, Endocr Rev 22: 706-17), and such analogs may be employed in connection with the present invention. Various strategies, including elongation of the COOH-terminal end of the insulin B-chain and engineering of fatty acid-acylated insulins with substantial affinity for albumin are used to generate longer-acting insulin analogs. However, in vivo treatments with available longer-acting insulin compounds still result in a high frequency of hypo- and hyperglycemic excursions and modest reduction in HbA1c. Accordingly, development of a truly long-acting and stable human insulin analog still remains an important task.

Functional analogs of insulin that may be employed in accordance with the invention include rapid acting analogs such as lispro, aspart and glulisine, which are absorbed rapidly (<30 minutes) after subcutaneous injection, peak at one hour, and have a relatively short duration of action (3 to 4 hours). In addition, two long acting insulin analogs have been developed: glargine and detemir, and which may be employed in connection with the invention. The long acting insulin analogs have an onset of action of approximately two hours and reach a plateau of biological action at 4 to 6 hours, and may last up to 24 hours.

Thus, in one embodiment, the insulin amino acid sequence may contain the A and/or B chain of lispro (also known as HUMALOG, Eli Lilly). Insulin lispro differs from human insulin by the substitution of proline with lysine at position 28 and the substitution of lysine with proline at position 29 of the insulin B chain. Although these modifications do not alter receptor binding, they help to de-stabilize insulin dimers and hexamers, allowing active monomeric insulin to be released quicker following preprandial injections. However, it is still necessary to formulate these insulins such that they exist in solution as dimers and multimers to provide long-term stability. Thus in some embodiments, the A and B chain sequences of lispro are used in connection with a fusion protein strategy described herein for rapid action. In other embodiments, lispro is co-formulated with a sustained release insulin fusion described herein.

In another embodiment, the insulin amino acid sequence may contain an A and/or B chain of aspart (also known as NOVOLOG, Novo Nordisk). Insulin aspart is designed with the single replacement of the amino acid proline by aspartic acid at position 28 of the human insulin B chain. This modification helps block the formation for insulin hexamers, creating a faster acting insulin. Thus in some embodiments, the A and B chain sequences of aspart are used in connection with a fusion protein strategy described herein for rapid action. In other embodiments, aspart is co-formulated with a sustained release insulin fusion described herein.

In yet another embodiment, the insulin amino acid sequence may contain an A and/or B chain of glulisine (also known as APIDRA, Sanofi-Aventis). Insulin glulisine is a short acting analog created by substitution of asparagine at position 3 by lysine and lysine at position 29 by glutamine of human insulin B chain. Insulin glulisine has more rapid onset of action and shorter duration of action compared to regular human insulin. Thus in some embodiments, the A and B chain sequences of glulisine are used in connection with a fusion protein strategy described herein. In other embodiments, glulisine is co-formulated with a sustained release insulin fusion described herein.

In another embodiment, the insulin amino acid sequence may contain an A and/or B chain of glargine (also known as LANTUS, Sanofi-Aventis). LANTUS has delayed absorption due to its acidic pH that causes microprecipitate formation of insulin crystals in the presence of neutral physiologic pH. Insulin glargine differs from human insulin in that the amino acid asparagine at position 21 of the A chain is replaced by glycine and two arginines are added to the C-terminus of the B-chain. Compared with bedtime neutral protamine Hagedorn (NPH) insulin (an intermediate acting insulin), insulin glargine is associated with less nocturnal hypoglycemia in patients with type 2 diabetes. Thus in some embodiments, the A and B chain sequences of insulin glargine are used in connection with a fusion protein strategy described herein for sustained release. In other embodiments, insulin glargine is co-formulated with a rapid action insulin fusion described herein.

In yet another embodiment, the insulin amino acid sequence may contain an A and/or B chain from detemir (also known as LEVEMIR, Novo Nordisk). Insulin detemir is a soluble (at neutral pH) long-acting insulin analog, in which the amino acid threonine at B30 is removed and a 14-carbon, myristoyl fatty acid is acetylated to the epsilon-amino group of LysB29. After subcutaneous injection, detemir dissociates, thereby exposing the free fatty acid which enables reversible binding to albumin molecules. So at steady state, the concentration of free unbound insulin is greatly reduced resulting in stable plasma glucose levels. Thus in some embodiments, the A and B chain sequences of detemir are used in connection with a fusion protein strategy described herein for sustained release. In other embodiments, detemir is co-formulated with a rapid action insulin fusion described herein.

In some embodiments, the insulin amino acid sequence may be a single-chain insulin analog (SIA) (e.g. as described in U.S. Pat. No. 6,630,438 and WO 2008/019368, which are hereby incorporated by reference in their entirety). Single-chain insulin analogs encompass a group of structurally-related proteins wherein the A and B chains are covalently linked by a polypeptide linker. The polypeptide linker connects the C-terminus of the B chain to the N-terminus of the A chain. The linker may be of any length so long as the linker provides the structural conformation necessary for the SIA to have a glucose uptake and insulin receptor binding effect. In some embodiments, the linker is about 5-18 amino acids in length. In other embodiments, the linker is about 9-15 amino acids in length. In certain embodiments, the linker is about 12 amino acids long. In certain exemplary embodiments, the linker has the sequence KDDNPNLPRLVR (SEQ ID NO:26) or GAGSSSRRAPQT (SEQ ID NO:27). However, it should be understood that many variations of this sequence are possible such as in the length (both addition and deletion) and substitutions of amino acids without substantially compromising the effectiveness of the produced SIA in glucose uptake and insulin receptor binding activities. For example, several different amino acid residues may be added or removed from either end without substantially decreasing the activity of the produced SIA.

An exemplary single-chain insulin analog currently in clinical development is albulin (Duttaroy et al., 2005, Diabetes 54: 251-8). Albulin can be produced in yeast or in mammalian cells. It consists of the B and A chain of human insulin (100% identity to native human insulin) linked together by a dodecapeptide linker and fused to the NH2 terminals of the native human serum albumin. For expression and purification of albulin, a synthetic gene was constructed encoding a single-chain insulin containing the B- and A-chain of mature human insulin linked together by a dodecapeptide linker using four overlapping primers and PCR amplification. The resulting PCR product was ligated in-frame between the signal peptide of human serum albumin (HSA) and the NH2 terminus of mature HSA, contained within a pSAC35 vector for expression in yeast. In accordance with the present invention, the HSA component of abulin may be replaced with an amino acid sequence providing a sustained release as described herein.

Thus, in one aspect, the present invention provides pharmaceutical compositions comprising an amino acid sequence providing for improved therapeutic properties (e.g., a sustained release or reduced multimer formation), including, for example, an elastin-like peptide (ELP), and an insulin amino acid sequence. For example, in certain embodiments, the insulin is a mammalian insulin, such as human insulin or porcine insulin. In accordance with the invention, the amino acid sequence providing a sustained release or loss of multimer formation may be coupled (e.g., via recombinant fusion or chemical conjugation) to the insulin A chain, or B chain, or both. In some embodiments, the amino acid sequence that provides a slow absorption from the injection site is covalently bound to the insulin A chain. The insulin may be a proinsulin and comprise each of chains A, B, and C, or may contain a processed form, containing only chains A and B. In some embodiments, chains A and B are connected by a short linking peptide, to create a single chain insulin. The insulin may be a functional analog of human insulin, including functional fragments truncated or extended at the N-terminus and/or C-terminus (of either or both of chains A and B) by from 1 to 10 amino acids, including by 1, 2, 3, or about 5 amino acids. Functional analogs may contain from 1 to 10 amino acid insertions, deletions, and/or substitutions (collectively) with respect to the native sequence (e.g., SEQ ID NOs: 24 and 25), and in each case retaining the activity of the peptide. For example, the positions 3, 28, 29, and 30 of the insulin B chain can be substituted, as well as position 21 of the A chain, including with K3, G21, K28, D28, P29, Q29. In some embodiments, from one to five amino acids are added to the C-terminus of the B chain, or T30 of the B chain may be removed. Such alterations may optionally correspond to the sequences of lispro, aspart, glulisine, glargine and detemir. Thus, functional analogs may have 1, 2, 3, 4, or 5 amino acid insertions, deletions, and/or substitutions (collectively) with respect to the native sequence (which may contain chains A and B, or chains A, B, and C), and may support further rapid action or delayed action in some embodiments. Such activity may be confirmed or assayed using any available assay, including those described herein. In these or other embodiments, the insulin chains have an amino acid sequence having at least about 75%, 80%, 85%, 90%, 95%, or 98% identity with each of the native sequences for chains A and B (SEQ ID NOS:24 and 25). The determination of sequence identity between two sequences (e.g., between a native sequence and a functional analog) can be accomplished using any alignment tool, including Tatusova et al., Blast 2 sequences—a new tool for comparing protein and nucleotide sequences, FEMS Microbiol Lett. 174:247-250 (1999). The insulin component may contain additional chemical modifications known in the art.

In some embodiments, the present invention provides for combination therapy of basal insulin and prandial insulin, comprising any combination of rapid action and delayed action insulin therapies described herein, and where at least one such agent has a fusion partner providing for either loss of multimer formation or sustained release. In some embodiments, at least one agent is insulin glargine, lispro, aspart, glulisine, glargine, detemir, a single-chain insulin analog (SIA), and albulin.

To characterize the in vitro binding properties of an insulin analog or an amino acid sequence providing a sustained release-containing insulin analog, competition binding assays may be performed in various cell lines that express the insulin receptor (Jehle et al., 1996, Diabetologia 39: 421-432). For example, competition binding assays using CHO cells overexpressing the human insulin receptor may be employed. Insulin can also bind to the IGF-1 receptor with a lower affinity than the insulin receptor. To determine the binding affinity of an amino acid sequence providing a sustained release-containing insulin analog, a competition binding assay can be performed using ¹²⁵I-labeled IGF-1 in L6 cells.

The activities of insulin include stimulation of peripheral glucose disposal and inhibition of hepatic glucose production. The ability of an amino acid sequence providing a sustained release-containing insulin analog to mediate these biological activities can be assayed in vitro using known methodologies. For example, the effect of an amino acid sequence providing a sustained release-containing analog on glucose uptake in 3T3-L1 adipocytes can be measured and compared with that of insulin. Pretreatment of the cells with a biologically active analog will generally produce a dose-dependent increase in 2-deoxyglucose uptake. The ability of an amino acid sequence providing a sustained release-containing insulin analog to regulate glucose production may be measured in any number of cells types, for example, H4IIe hepatoma cells. In this assay, pretreatment with a biologically active analog will generally result in a dose-dependent inhibition of the amount of glucose released.

Amino Acid Sequences Providing Sustained Release and/or Loss of Multimer Formation

In some embodiments, the amino acid sequence providing sustained release comprises structural units that form hydrogen-bonds through protein backbone groups and/or side chain groups, and which may contribute hydrophobic interactions to matrix formation. In some embodiments, the amino acid side chains do not contain hydrogen bond donor groups, with hydrogen bonds being formed substantially through the protein backbone. Exemplary amino acids include proline, alanine, valine, glycine, and isoleucine, and similar amino acids. In some embodiments, the structural units are substantially repeating structural units, so as to create a substantially repeating structural motif, and substantially repeating hydrogen-bonding capability. In these and other embodiments, the amino acid sequence comprises at least 10%, at least 20%, at least 40%, or at least 50% proline, which may be positioned in a pattern. The pattern of proline may create a repeating β-turn structure, or form an extended conformation with little or no defined secondary structure (e.g., no beta sheets or alpha helices). In these embodiments, the fusion partner is defined as a random coil conformation. In this context, a pattern of proline residues means that at least 50% or at least 75% of the proline residues of the amino acid sequence are part of a definable unit or primary sequence. In still other embodiments, the amino acid sequence comprises amino acids with hydrogen-bond donor side chains, such as serine, threonine, and/or tyrosine. In some embodiments, the repeating sequence may contain from one to about four proline residues, with remaining residues independently selected from non-polar residues, such as glycine, alanine, leucine, isoleucine, and valine. Non-polar or hydrophobic residues may contribute hydrophobic interactions to the formation of the matrix.

The amino acid sequences in these embodiments may form a “gel-like” state upon injection at a temperature higher than the storage temperature. Exemplary sequences have repeating peptide units, and may be relatively unstructured at the lower temperature, and achieve a hydrogen-bonded, structured, state at the higher temperature.

In some embodiments, the amino acid sequence capable of forming the matrix at body temperature is a peptide having repeating units of from four to ten amino acids. The repeating unit may form one, two, or three hydrogen bonds in the formation of the matrix. In certain embodiments, the amino acid sequence capable of forming the matrix at body temperature is an amino acid sequence of silk, elastin, collagen, or keratin, or mimic thereof, or an amino acid sequence disclosed in U.S. Pat. No. 6,355,776, which is hereby incorporated by reference.

In certain embodiments, the amino acid sequence is an Elastin-Like-Protein (ELP) sequence. The ELP sequence comprises or consists of structural peptide units or sequences that are related to, or mimics of, the elastin protein. The ELP sequence is constructed from structural units of from three to about twenty amino acids, or in some embodiments, from four to ten amino acids, such as four, five or six amino acids. The length of the individual structural units may vary or may be uniform. Exemplary structural units include units defined by SEQ ID NOS: 1-12 (below), which may be employed as repeating structural units, including tandem-repeating units, or may be employed in some combination. Thus, the ELP may comprise or consist essentially of structural unit(s) selected from SEQ ID NOS: 1-12, as defined below.

In some embodiments, including embodiments in which the structural units are ELP units, the amino acid sequence comprises or consists essentially of from about 50 to about 500 structural units, or in certain embodiments about 50 to about 200 structural units, or in certain embodiments from about 80 to about 180 structural units, or from about 100 to about 150 structural units, such as one or a combination of units defined by SEQ ID NOS: 1-12. Thus, the structural units collectively may have a length of from about 50 to about 2000 amino acid residues, or from about 100 to about 800 amino acid residues, or from about 200 to about 700 amino acid residues, or from about 400 to about 600 amino acid residues, or from about 400 to about 1000 amino acid residues, or from about 500 to about 1000 amino acid residues, or from about 600 to about 1000 amino acid residues, or from about 700 to about 1000 amino acid residues.

The amino acid sequence may exhibit a visible and reversible inverse phase transition with the selected formulation. That is, the amino acid sequence may be structurally disordered and highly soluble in the formulation below a transition temperature (Tt), but exhibit a sharp (2-3° C. range) disorder-to-order phase transition when the temperature of the formulation is raised above the Tt. In addition to temperature, length of the amino acid polymer, amino acid composition, ionic strength, pH, pressure, temperature, selected solvents, presence of organic solutes, and protein concentration may also affect the transition properties, and these may be tailored in the formulation for the desired absorption profile. Absorption profile can be easily tested by determining plasma concentration or activity of the insulin amino acid sequence over time.

In certain embodiments, the ELP component(s) may be formed of structural units, including but not limited to:
(a) the tetrapeptide Val-Pro-Gly-Gly, or VPGG (SEQ ID NO: 1);
(b) the tetrapeptide Ile-Pro-Gly-Gly, or IPGG (SEQ ID NO: 2);
(c) the pentapeptide Val-Pro-Gly-X-Gly (SEQ ID NO: 3), or VPGXG, where X is any natural or non-natural amino acid residue, and where X optionally varies among polymeric or oligomeric repeats;
(d) the pentapeptide Ala-Val-Gly-Val-Pro, or AVGVP (SEQ ID NO: 4);
(e) the pentapeptide Ile-Pro-Gly-X-Gly, or IPGXG (SEQ ID NO: 5), where X is any natural or non-natural amino acid residue, and where X optionally varies among polymeric or oligomeric repeats;
(f) the pentapeptide Ile-Pro-Gly-Val-Gly, or IPGVG (SEQ ID NO: 6);
(g) the pentapeptide Leu-Pro-Gly-X-Gly, or LPGXG (SEQ ID NO: 7), where X is any natural or non-natural amino acid residue, and where X optionally varies among polymeric or oligomeric repeats;
(h) the pentapeptide Leu-Pro-Gly-Val-Gly, or LPGVG (SEQ ID NO: 8);
(i) the hexapeptide Val-Ala-Pro-Gly-Val-Gly, or VAPGVG (SEQ ID NO: 9);
(j) the octapeptide Gly-Val-Gly-Val-Pro-Gly-Val-Gly, or GVGVPGVG (SEQ ID NO: 10);
(k) the nonapeptide Val-Pro-Gly-Phe-Gly-Val-Gly-Ala-Gly, or VPGFGVGAG (SEQ ID NO: 11); and
(l) the nonapeptides Val-Pro-Gly-Val-Gly-Val-Pro-Gly-Gly, or VPGVGVPGG (SEQ ID NO: 12).

Such structural units defined by SEQ ID NOs: 1-12 may form structural repeat units, or may be used in combination to form an ELP. In some embodiments, the ELP component is formed entirely (or almost entirely) of one or a combination of (e.g., 2, 3 or 4) structural units selected from SEQ ID NOs: 1-12. In other embodiments, at least 75%, or at least 80%, or at least 90% of the ELP component is formed from one or a combination of structural units selected from SEQ ID NOs: 1-12, and which may be present as repeating units.

In certain embodiments, the ELP comprises repeat units, including tandem repeating units, of Val-Pro-Gly-X-Gly (SEQ ID NO: 3), where X is as defined above, and where the percentage of Val-Pro-Gly-X-Gly (SEQ ID NO: 3) units taken with respect to the entire ELP component (which may comprise structural units other than VPGXG (SEQ ID NO: 3)) is greater than about 50%, or greater than about 75%, or greater than about 85%, or greater than about 95% of the ELP. The ELP may contain motifs of SEQ ID NO: 3, with the guest residue X varying among at least 2 or at least 3 of the units in the motif. The guest residues may be independently selected, such as from non-polar or hydrophobic residues, such as the amino acids V, I, L, A, G, and W (and may be selected so as to retain a desired inverse phase transition property).

In some embodiments, the ELP may form a β-turn structure. Exemplary peptide sequences suitable for creating a β-turn structure are described in International Patent Application PCT/US96/05186, which is hereby incorporated by reference in its entirety. For example, the fourth residue (X) in the sequence VPGXG (SEQ ID NO: 3), can be altered without eliminating the formation of a β-turn.

The structure of exemplary ELPs may be described using the notation ELPk [XiYj-n], where k designates a particular ELP repeat unit, the bracketed capital letters are single letter amino acid codes and their corresponding subscripts designate the relative ratio of each guest residue X in the structural units (where applicable), and n describes the total length of the ELP in number of the structural repeats. For example, ELP1 [V5A2G3-10] designates an ELP component containing 10 repeating units of the pentapeptide VPGXG (SEQ ID NO: 3), where X is valine, alanine, and glycine at a relative ratio of about 5:2:3; ELP1 [K1V2F1-4] designates an ELP component containing 4 repeating units of the pentapeptide VPGXG (SEQ ID NO: 3), where X is lysine, valine, and phenylalanine at a relative ratio of about 1:2:1; ELP1 [K1V7F1-9] designates a polypeptide containing 9 repeating units of the pentapeptide VPGXG (SEQ ID NO: 3), where X is lysine, valine, and phenylalanine at a relative ratio of about 1:7:1; ELP1 [V-5] designates a polypeptide containing 5 repeating units of the pentapeptide VPGXG (SEQ ID NO:3), where X is valine; ELP1 [V-20] designates a polypeptide containing 20 repeating units of the pentapeptide VPGXG (SEQ ID NO: 3), where X is valine; ELP2 [5] designates a polypeptide containing 5 repeating units of the pentapeptide AVGVP (SEQ ID NO: 4); ELP3 [V-5] designates a polypeptide containing 5 repeating units of the pentapeptide IPGXG (SEQ ID NO: 5), where X is valine; ELP4 [V-5] designates a polypeptide containing 5 repeating units of the pentapeptide LPGXG (SEQ ID NO: 7), where X is valine.

With respect to ELP, the Tt is a function of the hydrophobicity of the guest residue. Thus, by varying the identity of the guest residue(s) and their mole fraction(s), ELPs can be synthesized that exhibit an inverse transition over a broad range. Thus, the Tt at a given ELP length may be decreased by incorporating a larger fraction of hydrophobic guest residues in the ELP sequence. Examples of suitable hydrophobic guest residues include valine, leucine, isoleucine, phenylalanine, tryptophan and methionine. Tyrosine, which is moderately hydrophobic, may also be used. Conversely, the Tt may be increased by incorporating residues, such as those selected from: glutamic acid, cysteine, lysine, aspartate, alanine, asparagine, serine, threonine, glycine, arginine, and glutamine.

For polypeptides having a molecular weight >100,000, the hydrophobicity scale disclosed in PCT/US96/05186 (which is hereby incorporated by reference in its entirety) provides one means for predicting the approximate Tt of a specific ELP sequence. For polypeptides having a molecular weight <100,000, the Tt may be predicted or determined by the following quadratic function: Tt=M0+M1X+M2X2 where X is the MW of the fusion protein, and M0=116.21; M1=−1.7499; M2=0.010349.

The ELP in some embodiments is selected or designed to provide a Tt ranging from about 10 to about 37° C. at formulation conditions, such as from about 20 to about 37° C., or from about 25 to about 37° C., or from about 30 to about 37° C. In some embodiments, the transition temperature at physiological conditions (e.g., 0.9% saline) is from about 32 to 36° C. (or about or in the range of 35 to 36° C.), to take into account a slightly lower peripheral temperature.

In some embodiments, the amino acid sequence having hydrogen bonding capabilities is sufficiently short and/or less hydrophobic so as to effectively avoid multimer formation of the insulin, while not inducing the sustained release functionality. For example, when employing ELP fusions, the “fast acting” insulin may contain from about 5 to about 50 ELP units, such as from about 10 to about 30 ELP units, such as about 10, about 15, about 20, or about 25 ELP units, or about 30 ELP units.

In various embodiments, fusion partner providing fast action may have from 5 to 200 amino acids, such as from about 50 to about 150 amino acids, so as to inhibit insulin multimer (e.g., hexamer) formation. In some embodiments, the fusion partner has an extended conformation, which may form a random coil or conformation of repeating beta-turns, which may result from a pattern of proline residues in the amino acid sequence. In some embodiments, the sequence of the fusion partner contains less than about 35%, less than about 30%, or less than about 25%, or less than about 20% hydrophobic residues (excluding alanine, glycine, and proline as hydrophobic residues). For example, hydophobic residues in this context include leucine, isoleucine, valine, methionine, cysteine, tyrosine, phenylalanine, tryptophan, and histidine. The sequence that reduces or eliminates hexamer formation should not induce sustained release from the injection site, or exhibit a reverse phase transition at body temperature or below. The sequence should act to stabilize the insulin in monomeric or substantially monomeric form in a physiologically compatible solution.

In some embodiments, the fusion providing for rapid action contains from 1 to about 10 negatively charged amino acids such as, for example, glutamic acid and/or aspartic acid to increase the negative charge and reduce hydrophobicity of the fusion partner. Such residues may be inserted between ELP structural units in some embodiments. In some embodiments, the ELP is comprised of about 10 to about 25 ELP units (e.g., SEQ ID NOS:1-12), such as VPGXG units (SEQ ID NO: 3), where X is independently selected from V, G, and A where G and A are present in an approximately equivalent amount as guest residues (within about 1 or 2), and V is present at from about 2 to about 3 times more than G or A. Alternatively, the ELP units are AVGVP (SEQ ID NO:4). For example, exemplary fusion partner for rapid action insulin is exemplified by SEQ ID NO:16 (FIG. 18) or SEQ ID NO:17 (FIG. 20). In other embodiments, the ELP is comprised of about 10 to about 25 VPGXG units (SEQ ID NO: 3), where X is independently selected from V, G, and A where G and A are present in an approximately equivalent amount as the guest residue (within about 1 or 2), and V is present at from about 2 to about 3 times less than G or A. For example, an exemplary fusion protein for rapid action insulin is exemplified by SEQ ID NO:18 (FIG. 22) or SEQ ID NO:19 (FIG. 24) or SEQ ID NO:21 (FIG. 28) or SEQ ID NO:22 (FIG. 30). In other embodiments, the ELP is comprised of about 10 to about 25 VPGXG units (SEQ ID NO: 3) as above, where positively charged residues are inserted between a portion of the ELP units, for example, in from about 2 to about 6 locations (e.g., about 3, 4, or 5 locations). An exemplary fusion partner according to these embodiments is shown in FIG. 26 (SEQ ID NO:20). Alternatively, the positively charged residues can be positioned as guest residues within the ELP units, inserted at another position in the ELP units.

In some embodiments, the fusion protein providing for sustained release and rapid action are produced together as a “breakaway” fusion, and released during processing by the action of protease (e.g., Trypsin). Such a design is shown in FIG. 33 (SEQ ID NO:23). In such embodiments, a 1:1 co-formulation (or other defined molar ratio) of rapid-action and sustained action insulins can be conveniently prepared. In various embodiments, the breakaway construct comprises from one to five rapid acting insulin units (e.g., one or two), with from one to five (e.g., one or two) sustained release insulin units, each being connected by a protease cleavage site.

In exemplary embodiments, the amino acid sequence capable of forming the hydrogen-bonded matrix at body temperature (e.g., resulting in sustained release) comprises [VPGXG]₉₀ (SEQ ID NO:28), where each X is selected from V, G, and A, and wherein the ratio of V:G:A may be about 5:3:2. For example, the amino acid sequence capable of forming the hydrogen-bonded matrix at body temperature may comprise [VPGXG]₁₂₀ (SEQ ID NO:29), where each X is selected from V, G, and A, and wherein the ratio of V:G:A may be about 5:3:2. 120 structural units of this ELP can provide a transition temperature of about 37° C. with about 5 to 15 mg/ml (e.g., about 10 mg/ml) of protein. At concentrations of about 40 to about 100 mg/mL the phase transition temperature is about 33, or about 34, or about 35, or about 36 degrees centigrade (just below body temperature), which allows for peripheral body temperature to be just less than 37° C.

Alternatively, the amino acid sequence capable of forming the matrix at body temperature comprises [VPGVG]₉₀ (SEQ ID NO:28), or [VPGVG]₁₂₀ (SEQ ID NO:29). As shown herein, 120 structural units of this ELP can provide a transition temperature at about 37° C. with about 0.005 to about 0.05 mg/ml (e.g., about 0.01 mg/ml) of protein.

Elastin-like-peptide (ELP) protein polymers and recombinant fusion proteins can be prepared as described in U.S. Patent Publication No. 2010/0022455, which is hereby incorporated by reference.

In other embodiments, the amino acid sequence capable of forming the matrix at body temperature may include a random coil or non-globular extended structure. For example, the amino acid sequence capable of forming the matrix at body temperature may comprise an amino acid sequence disclosed in U.S. Patent Publication No. 2008/0286808, WIPO Patent Publication No. 2008/155134, and U.S. Patent Publication No. 2011/0123487, each of which is hereby incorporated by reference. In some embodiments, the amino acid sequence capable of forming the matrix at body temperature may be predominantly composed of proline with one or more of serine, alanine, and glycine residues. In some embodiments, the amino acid sequence capable of forming the matrix at body temperature is 50%, or 60%, or 70%, or 75%, or 80%, or 90% of proline, serine, alanine, and glycine residues (collectively).

For example, in some embodiments the amino acid sequence comprises an unstructured recombinant polymer of at least 40 amino acids. For example, the unstructured polymer may be defined where the sum of glycine (G), aspartate (D), alanine (A), serine (S), threonine (T), glutamate (E) and proline (P) residues contained in the unstructured polymer, constitutes more than about 80% of the total amino acids. In some embodiments, at least 50% of the amino acids are devoid of secondary structure as determined by the Chou-Fasman algorithm. The unstructured polymer may comprise more than about 100, 150, 200 or more contiguous amino acids. In some embodiments, the amino acid sequence forms a random coil domain. In particular, a polypeptide or amino acid polymer having or forming “random coil conformation” substantially lacks a defined secondary and tertiary structure.

In various embodiments, the intended subject is human, and the body temperature is about 37° C., and thus the pharmaceutical composition is designed to provide a sustained release at this temperature. A slow release into the circulation with reversal of hydrogen bonding and/or hydrophobic interactions is driven by a drop in concentration as the product diffuses at the injection site, even though body temperature remains constant. In other embodiments, the subject is a non-human mammal, and the pharmaceutical composition is designed to exhibit a sustained release at the body temperature of the mammal, which may be from about 30 to about 40° C. in some embodiments, such as for certain domesticated pets (e.g., dog or cat) or livestock (e.g., cow, horse, sheep, or pig). Generally, the Tt is higher than the storage conditions of the formulation (which may be from 10 to about 25° C., or from 15 to 22° C.), such that the pharmaceutical composition remains in solution for injection.

In some embodiments, the slow release is affected by administering cold formulations (e.g. 2-15° C., or 2-10° C., or 2-5° C.) of the pharmaceutical compositions of the present invention. Accordingly, in some embodiments, cold formulations are provided. Cold formulations may be administered at from about 2 to about 3° C., about 2 to about 4° C., about 2 to about 5° C., about 2 to about 6° C., about 2 to about 7° C., about 2 to about 8° C., about 2 to about 10° C., about 2 to about 12° C., about 2 to about 14° C., about 2 to about 15° C., about 2 to about 16° C., about 2 to about 20° C., about 10 to about 25° C., or from 15 to 22° C.

### Sustained Release Profiles

In one aspect, the invention provides a sustained release (e.g., long-acting) pharmaceutical formulation. The formulation comprises a pharmaceutical composition for systemic administration, where the pharmaceutical composition comprises an insulin amino acid sequence and an amino acid sequence capable of forming a reversible matrix (i.e. an amino acid sequence providing sustained release) at the body temperature of a subject as described herein. The reversible matrix is formed from hydrogen bonds (e.g., intra- and/or intermolecular hydrogen bonds) as well as from hydrophobic contributions. The formulation further comprises one or more pharmaceutically acceptable excipients and/or diluents inducing the formation of the matrix upon administration. The matrix provides for a slow absorption to the circulation from an injection site. The sustained release, or slow absorption from the injection site, is due to a slow reversal of the matrix as the concentration dissipates at the injection site. Once product moves into the circulation, the formulation confers long half-life and improved stability. Thus, a unique combination of slow absorption and long half-life is achieved leading to a desirable PK profile with a shallow peak to trough ratio and/or long Tmax.

Specifically, the invention provides improved pharmacokinetics for basal insulin, including a relatively flat PK profile with a low ratio of peak to trough, and/or a long Tmax. The PK profile can be maintained with a relatively infrequent administration schedule, such as from one to eight injections per month in some embodiments. In some embodiments, the basal insulin has a PK profile suitable for once weekly or once daily injection, providing substantial biological action for at least about 15 hours, at least about 20 hours, or about 24 hours for daily versions, and at least about 4 days, at least about 5 days, at least about 6 days, or about 7 days for weekly versions.

In one aspect, the invention provides a sustained release pharmaceutical formulation. The formulation comprises a pharmaceutical composition for systemic administration, where the pharmaceutical composition comprises an insulin amino acid sequence and an amino acid sequence capable of forming a matrix at the body temperature of a subject. The reversible matrix is formed from hydrogen bonds (e.g., intra- and/or intermolecular hydrogen bonds) as well as from hydrophobic contributions. The formulation further comprises one or more pharmaceutically acceptable excipients and/or diluents inducing the formation of the matrix upon administration. The matrix provides for a slow absorption to the circulation from an injection site, and without being bound by theory, this slow absorption is due to the slow reversal of the matrix as protein concentration decreases at the injection site. The slow absorption profile provides for a flat PK profile, as well as convenient and comfortable administration regimen. For example, in various embodiments, with either daily or weekly sustained release products, the plasma concentration of the insulin amino acid sequence over the course of days (e.g., from 2 to about 60 days, or from about 4 to about 30 days) does not change by more than a factor of 10, or by more than a factor of about 5, or by more than a factor of about 4 or by a factor of about 3. Generally, this flat PK profile is seen over a plurality of (substantially evenly spaced) administrations, such as at least 2, at least about 5, or at least about 10 administrations of the formulation. In some embodiments, the slow absorption is exhibited by a Tmax (time to maximum plasma concentration) of greater than about 1 hour, greater than about 2 hours, greater than about 5 hours, or greater than about 10 hours.

The sustained release, or slow absorption from the injection site, is controlled by the amino acid sequence capable of forming a hydrogen-bonded matrix at the body temperature of the subject, as well as the components of the formulation.

The formulation comprises one or more pharmaceutically acceptable excipients and/or diluents inducing the formation of the matrix upon administration. For example, such excipients include salts, and other excipients that may act to stabilize hydrogen bonding. Exemplary salts include alkaline earth metal salts such as sodium, potassium, and calcium. Counter ions include chloride and phosphate. Exemplary salts include sodium chloride, potassium chloride, magnesium chloride, calcium chloride, and potassium phosphate.

The protein concentration in the formulation is tailored to drive, along with the excipients, the formation of the matrix at the temperature of administration. For example, higher protein concentrations help drive the formation of the matrix, and the protein concentration needed for this purpose varies depending on the ELP series used. For example, in embodiments using an ELP1-120, or amino acid sequences with comparable transition temperatures, the protein is present in the range of about 10 mg/mL to about 200 mg/mL, or is present in the range of about 50 mg/mL to about 150 mg/mL. In exemplary embodiments, the invention provides a sustained release pharmaceutical formulation that comprises a therapeutic agent, the therapeutic agent (e.g., a peptide or protein therapeutic agent) comprising an insulin amino acid sequence and an amino acid sequence comprising [VPGXG]₉₀ (SEQ ID NO:28), or [VPGXG]₁₂₀ (SEQ ID NO:29), where each X is selected from V, G, and A. V, G, and A may be present at a ratio of about 5:3:2.

The pharmaceutical composition is formulated at a pH, ionic strength, and generally with excipients sufficient to drive the formation of the matrix at body temperature (e.g., 37° C., or at from 32 to 36° C. in some embodiments). The pharmaceutical composition is generally prepared such that it does not form the matrix at storage conditions. Storage conditions are generally less than the transition temperature of the formulation, such as less than about 32° C., or less than about 30° C., or less than about 27° C., or less than about 25° C., or less than about 20° C., or less than about 15° C. For example, the formulation may be isotonic with blood or have an ionic strength that mimics physiological conditions. For example, the formulation may have an ionic strength of at least that of 25 mM Sodium Chloride, or at least that of 30 mM Sodium chloride, or at least that of 40 mM Sodium Chloride, or at least that of 50 mM Sodium Chloride, or at least that of 75 mM Sodium Chloride, or at least that of 100 mM Sodium Chloride, or at least that of 150 mM Sodium Chloride. In certain embodiments, the formulation has an ionic strength less than that of about 0.9% saline. In some embodiments, the formulation comprises two or more of calcium chloride, magnesium chloride, potassium chloride, potassium phosphate monobasic, sodium chloride, and sodium phosphate dibasic. In certain embodiments, the formulation may comprise about 50 mM histidine, or about 40 mM histidine, or about 30 mM histidine, or about 25 mM histidine, or about 20 mM histidine, or about 15 mM histidine. The liquid formulation may comprise about 100 mM Sodium Chloride and about 20 mM histidine and can be stored refrigerated or at room temperature. The salt concentration can be adjusted to provide isotonicity at the site of injection.

The formulation can be packaged in the form of pre-dosed pens or syringes for administration once per week, twice per week, or from one to eight times per month, or alternatively filled in conventional vial and the like.

Other formulation components for achieving the desired stability, for example, may also be employed. Such components include one or more amino acids or sugar alcohol (e.g., mannitol), preservatives, and buffering agents, and such ingredients are well known in the art.

In some embodiments, the formulation is administered about once daily, and may be administered subcutaneously or intramuscularly.

### Rapid Onset Profile

In one aspect, the invention provides a rapid action insulin formulation. The formulation comprises a pharmaceutical composition for systemic administration, where the pharmaceutical composition comprises an insulin amino acid sequence and an amino acid sequence capable of inhibiting insulin multimer formation as described. The monomeric state is stabilized in the formulation, allowing rapid onset of insulin action, and being suitable for providing prandial insulin.

Specifically, the rapid onset insulin exhibits glucose lowering effects within 1 hour, and in various embodiments within about 30 minutes, or within about 15 minutes, or within about 10 minutes, or within about 5 minutes. Maximum effects are exhibited by about one hour, or by about 30 minutes in some embodiments. The effects are not substantially sustained beyond about 6 hours, or beyond about 5 hours, or beyond about 4 hours, thereby making the agent and formulation suitable for providing prandial insulin, and complementing a sustained (e.g., basal) insulin product.

The formulation comprises one or more pharmaceutically acceptable excipients and/or diluents which stabilize the monomeric state of the rapid onset insulin, and in some embodiments, are the same conditions that allow for in vivo phase transition of the sustained action insulin, thereby allowing for a practical co-formulation. For example, such excipients include salts, including alkaline earth metal salts such as sodium, potassium, and calcium. Counter ions include chloride and phosphate. Exemplary salts include sodium chloride, potassium chloride, magnesium chloride, calcium chloride, and potassium phosphate.

The protein concentration in the formulation may be about 10 mg/mL to about 200 mg/mL, or is present in the range of about 50 mg/mL to about 150 mg/mL. The pharmaceutical composition may be present in the range of about 10 mg/mL to about 50 mg/mL. Exemplary rapid onset insulin constructs include SEQ ID NOS:16, 17, 18, 19, 20, 21, and 22, or similar constructs having the same or similar molecular weights (e.g., within about 10%). In some embodiments, the formulation comprises a 1:1 molar ratio of basal and rapid-acting insulins.

The formulation can be packaged in the form of pre-dosed pens or syringes, or conventional vials for administration once, twice, or three times daily.

Other formulation components for achieving the desired stability, for example, may also be employed. Such components include one or more amino acids or sugar alcohol (e.g., mannitol), preservatives, and buffering agents, and such ingredients are well known in the art.

### Conjugation and Coupling

A recombinantly-produced fusion protein, in accordance with certain embodiments of the invention, includes an amino acid sequence providing sustained release or reduced multimer formation (e.g., ELP) and an insulin amino acid sequence associated with one another by genetic fusion. For example, the fusion protein may be generated by translation of a polynucleotide encoding an insulin amino acid sequence cloned in-frame with the amino acid sequence providing sustained release component.

In certain embodiments, the amino acid sequence of the fusion partner and insulin amino acid sequence can be fused using a linker peptide of various lengths to provide greater physical separation and allow more spatial mobility between the fused portions, and thus maximize the accessibility of the insulin amino acid sequence for binding to its receptor. The linker peptide may consist of amino acids that are flexible or more rigid. For example, a flexible linker may include amino acids having relatively small side chains, and which may be hydrophilic. Without limitation, the flexible linker may comprise glycine and/or serine residues. More rigid linkers may contain, for example, more sterically hindering amino acid side chains, such as (without limitation) tyrosine or histidine. The linker may be less than about 50, 40, 30, 20, 10, or 5 amino acid residues. The linker can be covalently linked to and between an insulin amino acid sequence and an amino acid sequence providing sustained release component, for example, via recombinant fusion.

The linker or peptide spacer may be protease-cleavable or non-cleavable. By way of example, cleavable peptide spacers include, without limitation, a peptide sequence recognized by proteases (in vitro or in vivo) of varying type, such as Tev, thrombin, factor Xa, plasmin (blood proteases), metalloproteases, cathepsins, and proteases found in other corporeal compartments. In some embodiments employing cleavable linkers, the fusion protein may be inactive, less active, or less potent as a fusion, which is then activated upon cleavage of the spacer in vivo. Alternatively, where the insulin amino acid sequence is sufficiently active as a fusion, a non-cleavable spacer may be employed. The non-cleavable spacer may be of any suitable type.

In other embodiments, the present invention provides chemical conjugates of an insulin amino acid sequence and the amino acid sequence providing sustained release reduced multimer functionality. The conjugates can be made by chemically coupling an amino acid sequence providing sustained release component to an insulin amino acid sequence, or by solid phase synthesis of desired sequences, by any number of methods well known in the art (See, e.g., Nilsson et al., 2005, Ann Rev Biophys Bio Structure 34: 91-118). In some embodiments, the chemical conjugate can be formed by covalently linking the insulin amino acid sequence to the amino acid sequence providing sustained release component, directly or through a short or long linker moiety, through one or more functional groups on the therapeutic proteinacious component, e.g., amine, carboxyl, phenyl, thiol or hydroxyl groups, to form a covalent conjugate. Various conventional linkers can be used, e.g., diisocyanates, diisothiocyanates, carbodiimides, bis(hydroxysuccinimide)esters, maleimide-hydroxysuccinimide esters, glutaraldehyde and the like.

Non-peptide chemical spacers can additionally be of any suitable type, including for example, by functional linkers described in Bioconjugate Techniques, Greg T. Hermanson, published by Academic Press, Inc., 1995, and those specified in the Cross-Linking Reagents Technical Handbook, available from Pierce Biotechnology, Inc. (Rockford, Ill.), the disclosures of which are hereby incorporated by reference, in their respective entireties. Illustrative chemical spacers include homobifunctional linkers that can attach to amine groups of Lys, as well as heterobifunctional linkers that can attach to Cys at one terminus, and to Lys at the other terminus.

In certain embodiments, relatively small ELP components (e.g., ELP components of less than about 30 kDa, 25 kDa, 20 kDa, 15 kDa, or 10 kDa), that do not transition at room temperature (or human body temperature, e.g., Tt >37° C.), are chemically coupled or crosslinked. For example, two relatively small ELP components, having the same or different properties, may be chemically coupled. Such coupling, in some embodiments, may take place in vivo, by the addition of a single cysteine residue at or around the C-terminus of the ELP. Such ELP components may each be fused to one or more insulin amino acid sequences, so as to increase activity or avidity at the target.

### Methods of Treating Diseases

In various embodiments, the treatment provides for sustained glycemic control. Glycemic control refers to the typical levels of blood sugar (glucose) in a person with diabetes mellitus. Many of the long-term complications of diabetes, including microvascular complications, result from many years of hyperglycemia. Good glycemic control is an important goal of diabetes care. Because blood sugar levels fluctuate throughout the day and glucose records are imperfect indicators of these changes, the percentage of hemoglobin which is glycosylated is used as a proxy measure of long-term glycemic control in research trials and clinical care of people with diabetes. In this test, the hemoglobin A1c or glycated hemoglobin (HbA1c) reflects average glucose values over the preceding 2-3 months.

In nondiabetic persons with normal glucose metabolism glycated hemoglobin levels are usually about 4-6% by the most common methods (normal ranges may vary by method). “Perfect glycemic control” indicates that glucose levels are always normal (e.g. about 70-130 mg/dl, or about 3.9-7.2 mmol/L) and indistinguishable from a person without diabetes. In reality, because of the imperfections of treatment measures, even “good glycemic control” describes blood glucose levels that average somewhat higher than normal much of the time. It is noted that what is considered “good glycemic control” varies by age and susceptibility of the patient to hypoglycemia. The American Diabetes Association has advocated for patients and physicians to strive for average glucose and hemoglobin A1c values below 200 mg/dl (11 mmol/l) and 8%. “Poor glycemic control” refers to persistently elevated blood glucose and glycosylated hemoglobin levels, which may range from, e.g., about 200-500 mg/dl (about 11-28 mmol/L) and about 9-15% or higher over months and years before severe complications occur.

In various embodiments, the pharmaceutical compositions of the present invention as described herein are used for the management and care of a patient having a pathology such as diabetes or hyperglycemia, or any other condition for which insulin administration is indicated for the purpose of combating or alleviating symptoms and complications of those conditions, including various metabolic disorders. Treating includes administering an agent, composition, or formulation of the present invention to prevent the onset of the symptoms or complications, alleviating the symptoms or complications, or eliminating the disease, condition, or disorder. The present methods include treatment of type 1 diabetes, i.e., a condition in which the body does not produce insulin and therefore cannot control the amount of sugar in the blood and type 2 diabetes, i.e., a condition in which the body does not use insulin normally and, therefore, cannot control the amount of sugar in the blood. In some embodiments, the patient has one or more complications of diabetes, including cardiovascular complications, or in some embodiments has metabolic disease and/or is characterized as being clinically obese or overweight. In some embodiments the patient has (at the start of the treatment regimen) an HbA1c above about 12%, above about 10%, above about 9%, or above about 8.5%, or above about 8%, or above about 7.5%, or above about 7.0, or above about 6.5.

Because the active agents have negligible or reduced interaction with the IGF Receptor in some embodiments, the active agents described herein are particularly suited for long term therapy, including treatment for a plurality of years (e.g., at least about 2 years, at least about 3 years, at least about 5 years, at least about 10 years, or more).

In some embodiments, the present invention provides for methods of administering combination therapy of basal insulin and prandial insulin, which may be administered separately or together. For example, in some embodiments, the patient receives a regimen of a rapid-acting and/or long-acting insulin, where at least one such agent has a fusion partner providing for either loss of multimer formation or sustained release. In some embodiments, the rapid-acting insulin that reduces multimer formation is provided as co-therapy with insulin glargine or detemir. That is, the rapid-acting insulin fusion is administered to a patient as described, where the patient is undergoing basal insulin therapy with insulin glargine or detemir. In other embodiments, the long-acting insulin that provides sustained release is provided as co-therapy with one or more of lispro, aspart, or glulisine. That is, the long-acting insulin fusion is administered to a patient as described, where the patient is undergoing prandial insulin therapy with insulin lispro, aspart, or glulisine.

In various embodiments, the present invention provides for combination therapies and/or co-formulations which comprise the pharmaceutical compositions described herein and other agents that are effective in treating diseases, such as those described above.

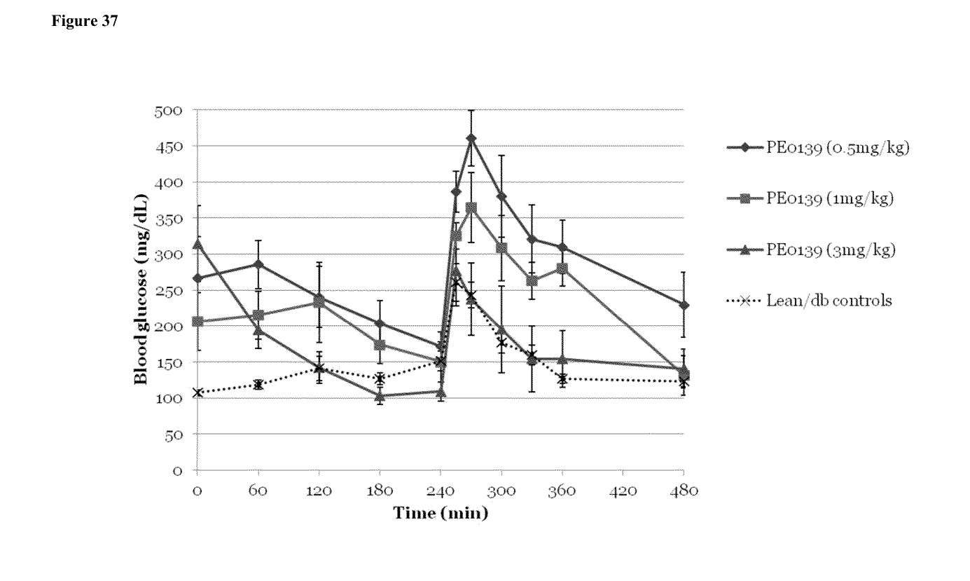

In some embodiments, the invention provides for combination or co-formulation (of sustained release insulin or prandial insulin) with a glucagon like receptor (GLP)-1 receptor agonist, such as GLP-1 (SEQ ID NO:30), exendin-4 (SEQ ID NO:31), or functional analogs and/or derivatives thereof as disclosed in U.S. Pat. No. 8,178,495, which is hereby incorporated by reference. In some embodiments, the GLP-1 is GLP-1 (A-B), wherein A is an integer from 1 to 7 and B is an integer from 38 to 45. In some embodiments, the GLP-1 is GLP-1 (7-36), or a functional analog thereof or GLP-1 (7-37), or a functional analog thereof. In some embodiments, the GLP-1 receptor agonist comprises a fusion partner that also allows for sustained release, for example, with a PK profile disclosed in US 2013/0090285, which is hereby incorporated by reference. Thus, the GLP-1 receptor agonist may have the sequence of SEQ ID NO:32. In some embodiments, the GLP-1 receptor agonist fusion partner is an ELP1-120, where the guest residues are valine, alanine, and glycine, at an approximate ratio of 5:2:3. As shown in FIGS. 37 to 41, such co-formulations provide for synergistic effects, and may allow for lower doses of both agents to be administered.

In another embodiment, the invention provides for combination or co-formulation with GLP-2, GIP, glucagon, and oxyntomodulin or functional analogs and/or derivatives thereof. Functional analogs may contain from 1 to 10 amino acid insertions, deletions, and/or substitutions (collectively) with respect to the native sequence.

In various embodiments, the combination therapies and/or co-formulations comprise two or more fusion proteins with, for example, ELP or a matrix-forming components as described herein. In some embodiments, the ELP comprises at least 60 units, or 90 units, or 120 units, or 180 units of VPGXG (SEQ ID NO: 3), where X is an independently selected amino acid. In various embodiments, X is V, G, or A at a ratio of 5:3:2, or K, V, or F at a ratio of 1:2:1, or K, V, or F at a ratio of 1:7:1, or V.

In various embodiments, compositions comprising a sustained release form of insulin, which optionally comprise a rapid onset form of insulin, are administered once a day before breakfast.

### EXAMPLES

### Example 1

### Sustained Release Insulin Constructs

Human proinsulin was genetically fused to the ELP1-120 biopolymer and expressed in the soluble fraction of E. coli. Following purification enzymatic processing of the proinsulin moiety into mature insulin the fusion protein was tested for glucose lowering in a normal mouse model and compared with insulin alone. The insulin ELP fusion showed glucose lowering similar to insulin. In addition the lowering effect of the fusion protein was shown to extend over a longer duration than that of insulin in the model.

### Insulin Fusion Construction

Human proinsulin consists of the B and A chains linked together with the 31 amino acid C peptide (FIGS. 1A and 1B). Once disulfides are formed between the B and A chains the proinsulin is converted into mature insulin in vivo by removal of the C peptide by a trypsin/carboxypeptidase B-like system. This peptide processing can be replicated in vitro using recombinant trypsin and carboxypeptidase B. Since the fusion is expressed in the soluble fraction of E. coli no refolding steps are necessary.

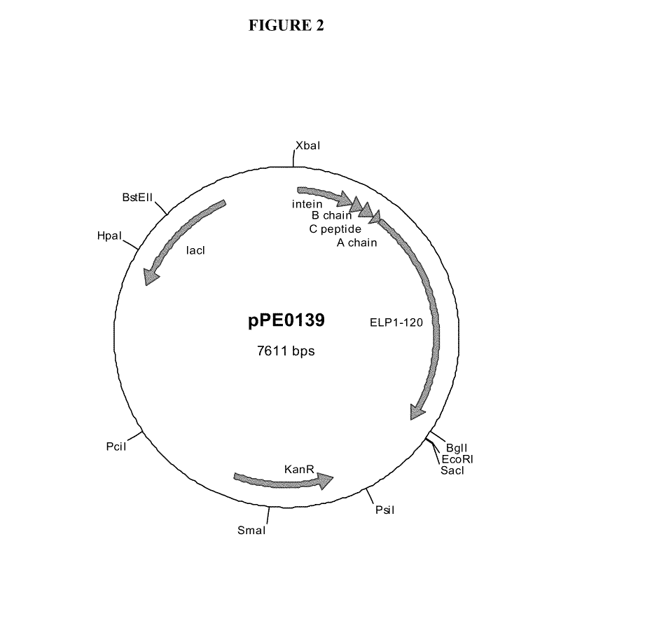

The proinsulin nucleotide sequence was synthesized and subcloned into pET based vector pPB1031 positioning it at the N-terminus of the ELP1-120 sequence to make plasmid pPE0139 (FIG. 2).

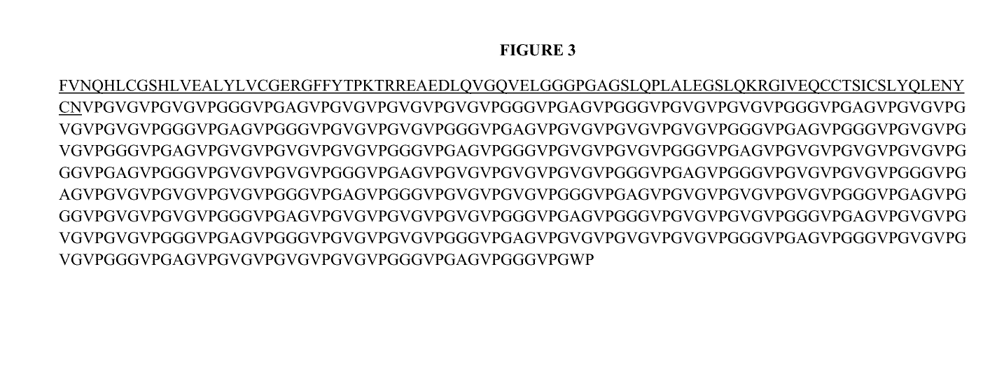

FIG. 3 shows the amino acid sequence of a proinsulin ELP1-120 fusion protein (SEQ ID NO:14). The proinsulin sequence (underlined) is fused to the ELP1-120 sequence. The amino acid sequence optionally includes an initiation methionine residue at the N terminus.

### Fermentation

Insulin ELP fusion plasmid pPE0139 was expressed in the intracellular fraction of E. coli under control of the T7 promoter in a fed-batch fermentation process. The glycerol cell stock was expanded using a two-stage shake flask seed train in semi-defined, animal-free medium (ECPM+Proline) with glycerol as the primary carbon source and yeast extract as the primary nitrogen source. After sufficient cell density was achieved in the seed train, the culture was transferred to a fermentor containing the same medium as the seed train. Process parameters (pH, temperature, dissolved oxygen) were maintained at set point via PID control. The culture grew until it reached stationary phase whereupon a glycerol/yeast extract/magnesium sulfate feed was initiated. The culture was maintained under carbon limitation and induction of the promoter was achieved using IPTG. At the end of the fermentation, the culture was centrifuged to separate the biomass containing the Insulin ELP fusion from the spent medium. The cell paste was stored at −70° C. until subsequent purification.

### Purification of Proinsulin ELP

Frozen cell paste was resuspended in lysis buffer containing 2M Urea (for dissociation of Proinsulin ELP) and mixed until homogenous. Lysis was achieved using a microfluidizer to disrupt the cell membranes and then initial clarification of the lysate was performed by centrifugation. A two stage tangential flow filtration (TFF) system was used to further clarify and concentrate the product. The Proinsulin ELP fusion-containing solution was adjusted with buffer concentrate to 1M sodium chloride and passed over a hydrophobic interaction chromatography (HIC) column (e.g. TOSOH PPG 600M) as a capture step and host cell contaminants were washed away. The product was eluted using a gradient to fractionate any product-related impurities (e.g. degraded species). TFF buffer exchange was performed on the selected fractions to remove residual salt prior to enzymatic conversion of the Proinsulin ELP to Insulin ELP.

### Enzymatic Processing of Proinsulin ELP Fusion

Purified Proinsulin ELP1-120 was converted to Insulin ELP through enzymatic digestion of the C-peptide using recombinant Trypsin and Carboxypeptidase B, at a ratio of 0.05 μg/mg Proinsulin and 2.0 μg/mg Proinsulin ELP, respectively. The reaction was incubated at room temperature (20-25° C.) for 3 to 4 hours until the C-peptide was fully cleaved to yield mature Insulin ELP.

### Purification of Insulin ELP

Following conversion to Insulin ELP, the residual enzymes and product variants were removed by reprocessing the Insulin ELP over the capture HIC column where the Insulin ELP bound to the column and the residual enzymes passed through. The eluted product fractions were diafiltered to remove NaCl and then purified by anion exchange (e.g. POROS 50 PI) under conditions where the product flows through the column and host cell contaminants are captured. Finally a Cation Exchange column (e.g. POROS 50 HS) was used to remove product variants, host cell protein, endotoxin and DNA. The eluted product was then concentrated and diafiltered into formulation buffer (e.g. 20 mM histidine, 110 mM NaCl, pH 7.5).

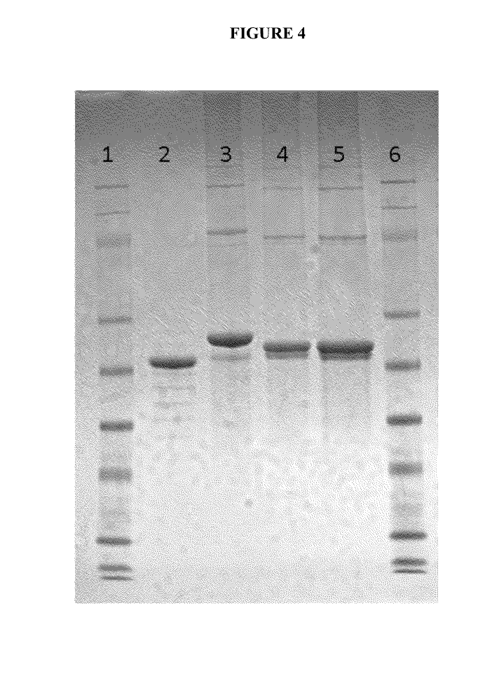

Non-reducing SDS-PAGE (FIG. 4) showed the expected decreased fusion protein molecular weight following enzymatic processing as the C-peptide was cleaved.

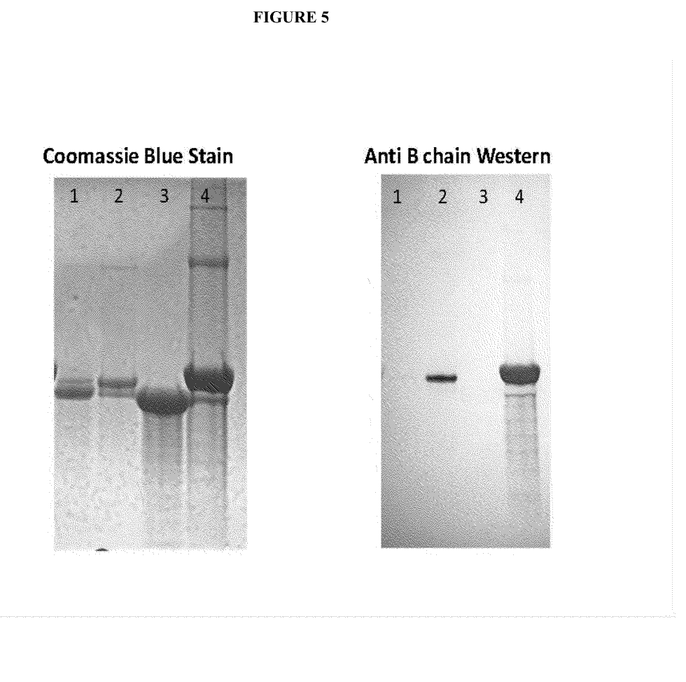

An anti-insulin B chain western blot (FIG. 5) was performed to confirm presence of both A and B chains fused to ELP. The data showed presence of B-chain under non-reducing conditions indicating disulfide bond formation between the A and B chains. Reduction of the fusion protein and disulfide bonds resulted in removal of B chain from the fusion.

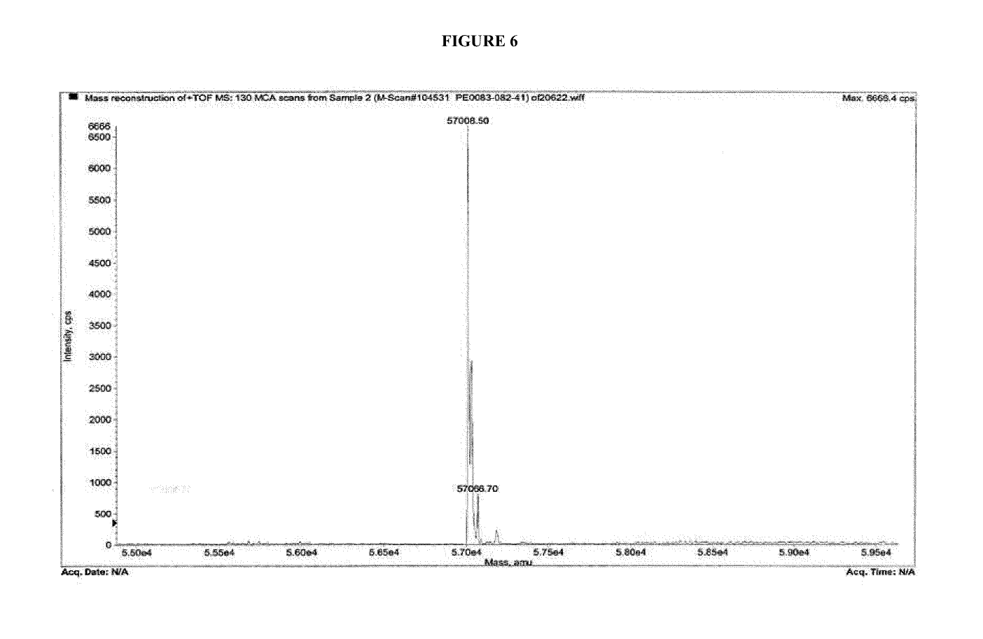

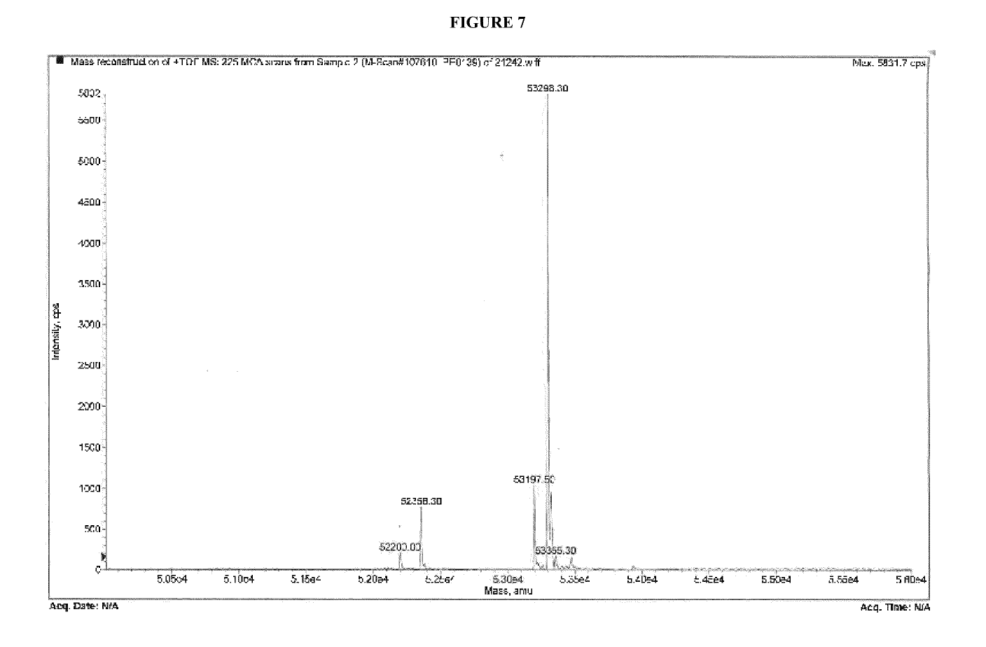

Electrospray ionization mass spectrometry confirmed the mass of Proinsulin ELP fusion (FIG. 6) and the mature Insulin ELP fusion following enzymatic removal of the C-peptide (FIG. 7). Additional salt adducts were present in both samples. Presence of disulfide bonds was confirmed using an Ellman's reagent assay. The absence of free thiols indicated disulfide bonds were formed.

### In Vivo Glucose Lowering

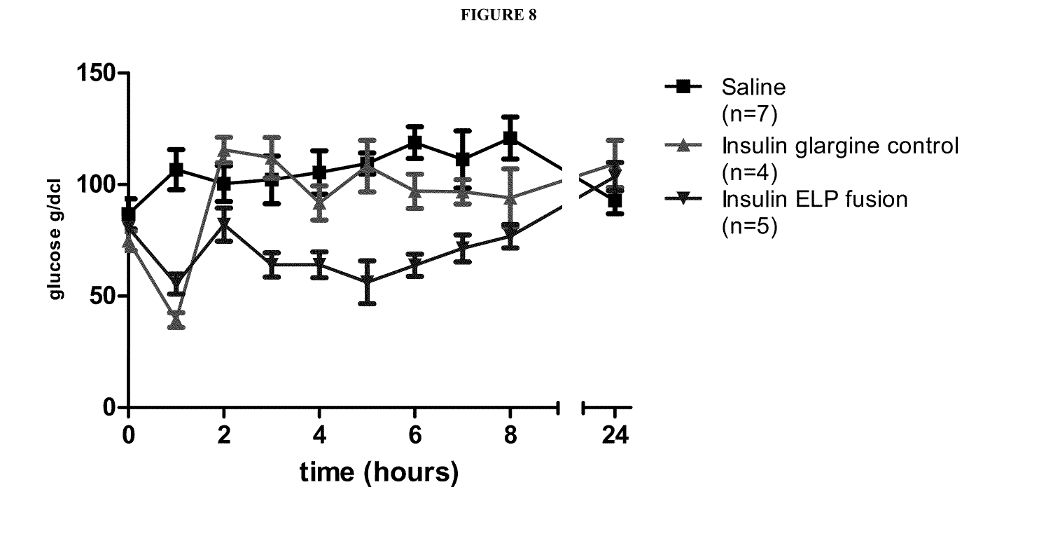

Normal mice were fasted overnight and injected subcutaneously with saline (negative control), 13 nmol/kg insulin glargine (positive control) or 35 nmol/kg insulin ELP fusion (INSUMERA). Blood glucose readings were taken prior to dosing and each hour after through 8 hours and 24 hours post dosing. Food was made available 1 hour post dose. FIG. 8 shows the blood glucose data (mean+−SE). The insulin ELP fusion shows significant blood glucose lowering versus the saline control. In addition the insulin ELP fusion showed a blood glucose lowering that extended farther (7 hours) than the insulin glargine control (2 hours).

### In Vivo Effects in a Type 1 Diabetes Model

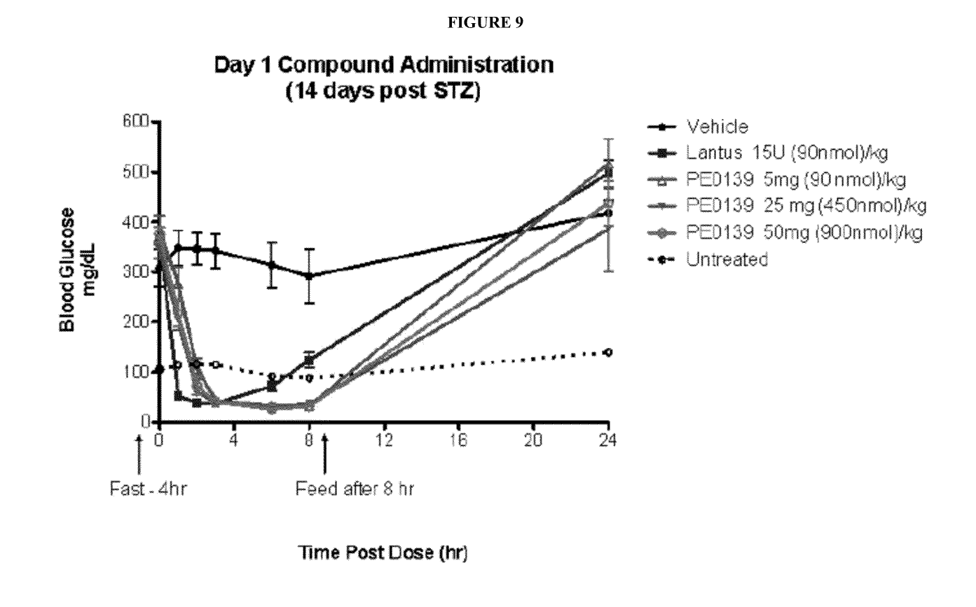

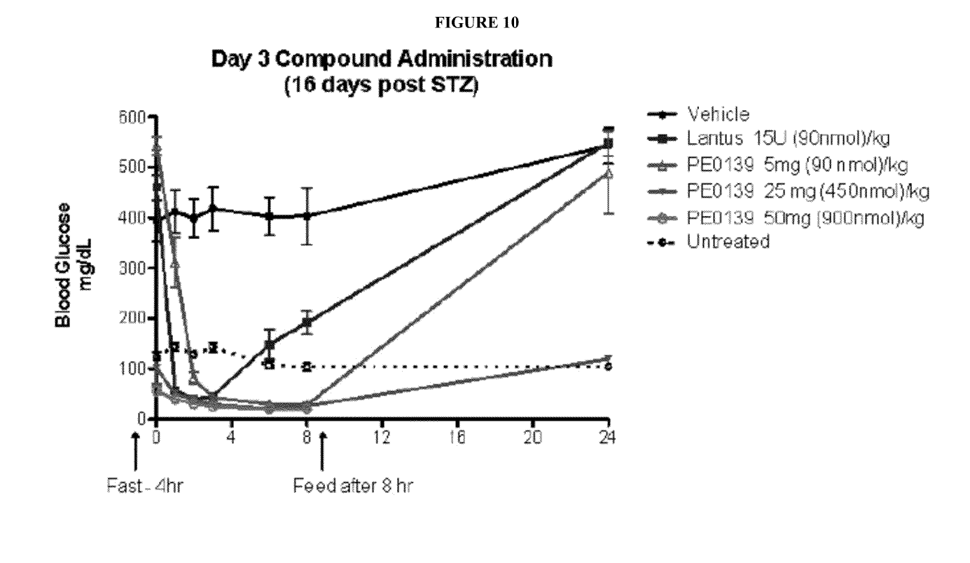

A ELP-Insulin fusion, INSUMERA (PE0139), was dosed in a diabetes mellitus type I (type 1 diabetes, T1DM) mouse model. Specifically, single dose data is shown in FIG. 9. The results demonstrated a greater duration of glucose lowering for INSUMERA, as compared to equimolar LANTUS (insulin glargine, SANOFI-AVENTIS) dosing. When the compounds were dosed on a daily regimen (FIG. 10), the results demonstrate the superiority of INSUMERA, as compared to LANTUS (insulin glargine, SANOFI-AVENTIS), with regards to activity and half-life.

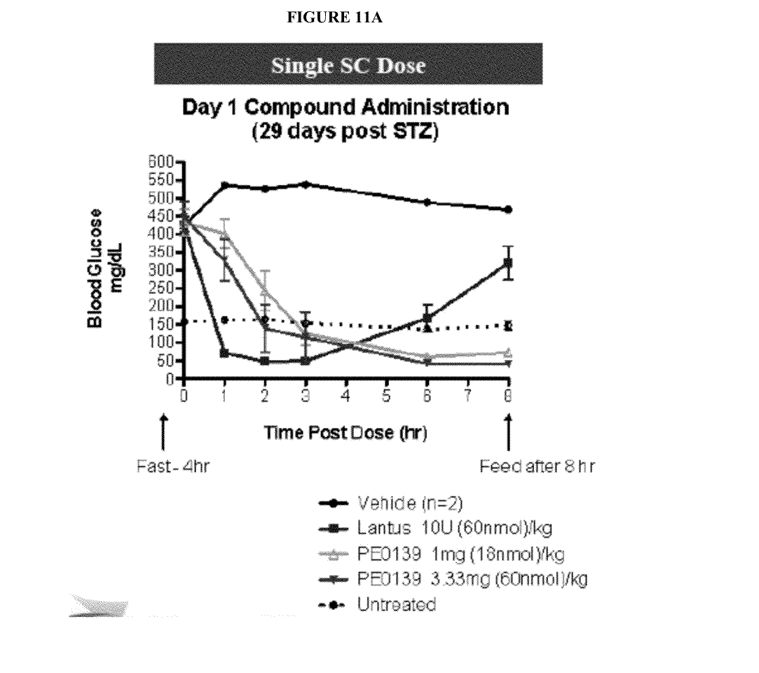

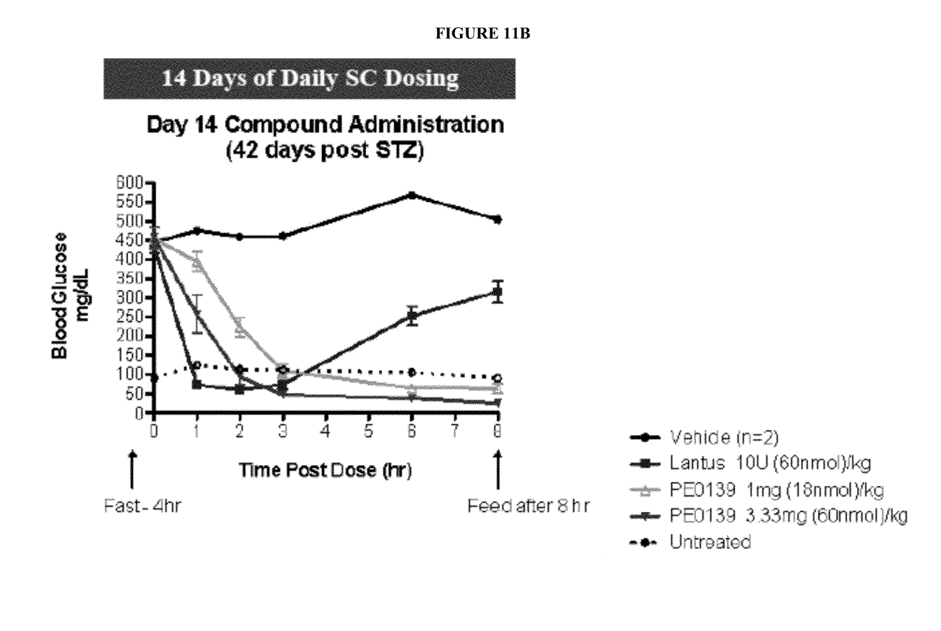

FIGS. 11A and 11B show INSUMERA (PE0139) low dose titration in a diabetes mellitus type 1 (type 1 diabetes, T1DM) mouse model as compared to LANTUS (insulin glargine, SANOFI-AVENTIS). FIG. 11A shows a single s.c. dose while FIG. 11B shows 14 days of daily s.c. dosing. In both cases, the more pronounced and sustained blood glucose lowering effect of INSUMERA is shown.

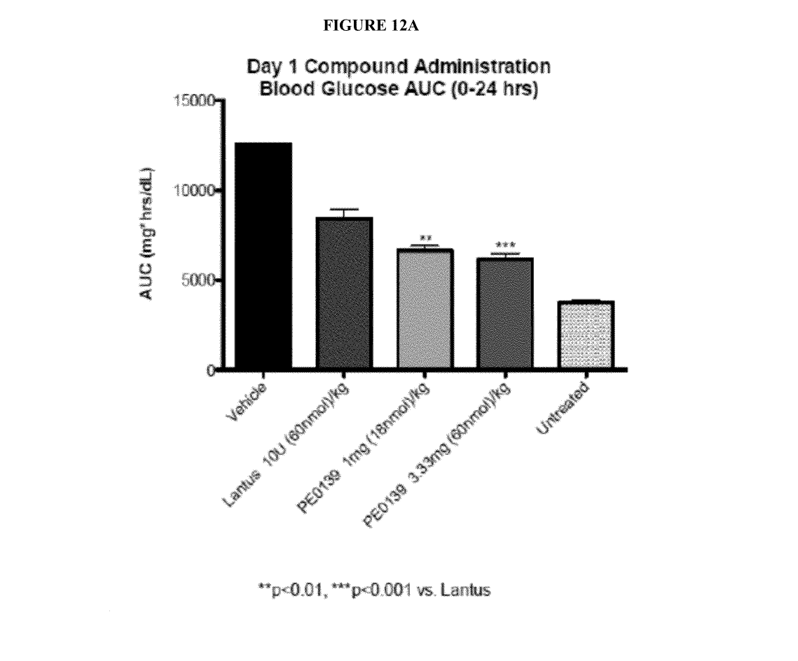

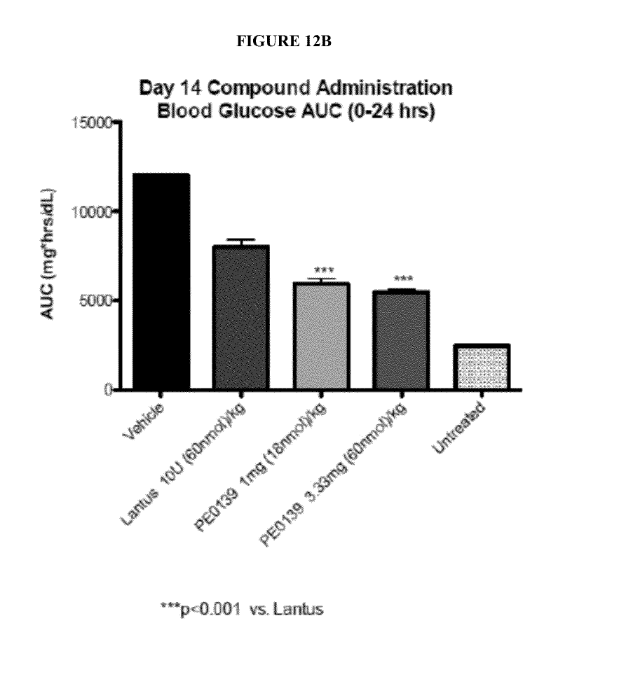

Studies were also conducted to determine the extent of glycemic control, a measure of the typical levels of blood sugar in a patient, of INSUMERA. FIGS. 12A and 11B shows that INSUMERA (PEW 39) has significantly increased glycemic control relative to LANTUS (insulin glargine, SANOFI-AVENTIS). A reduction of 27-39% is seen in area under the curve (AUC) blood glucose. FIG. 12A shows day 1 of compound administration and the blood glucose AUC at 0-24 hrs. FIG. 12B shows day 14 of compound administration and the blood glucose AUC at 0-24 hrs. INSUMERA reduced blood glucose AUC, more effectively than LANTUS, in both dosing regimes.

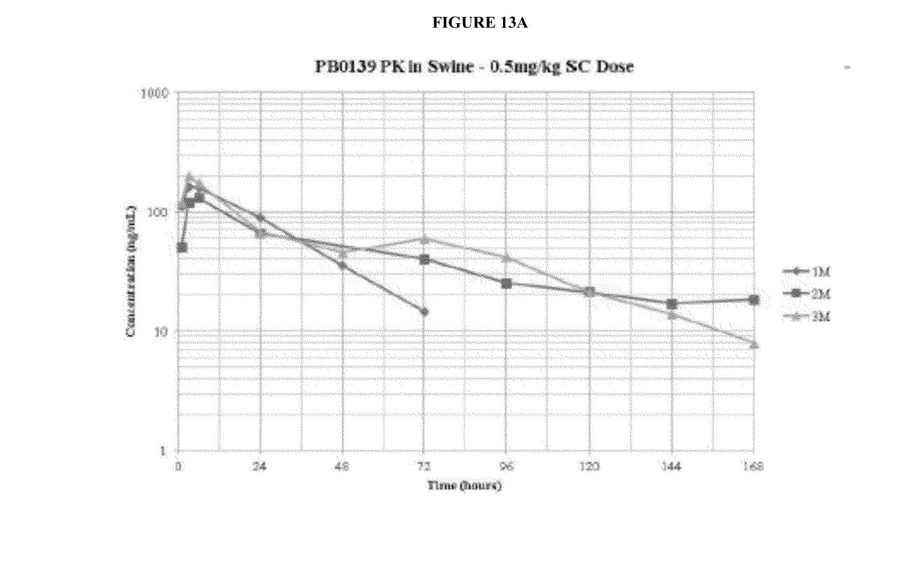

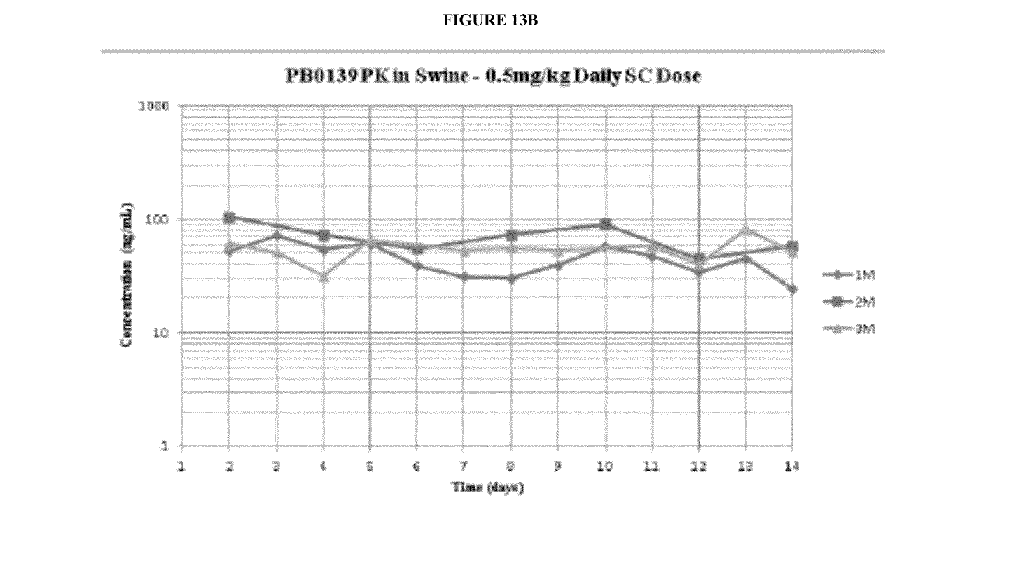

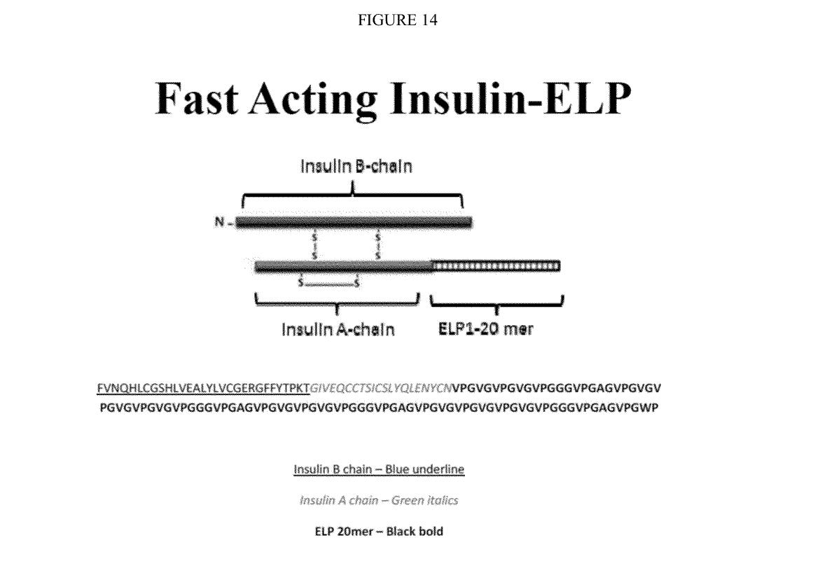

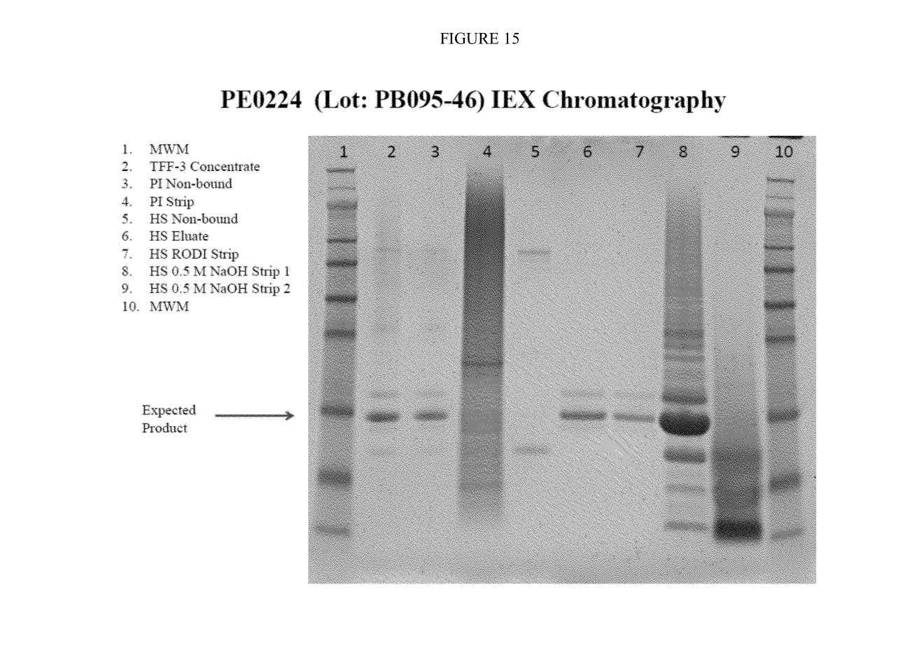

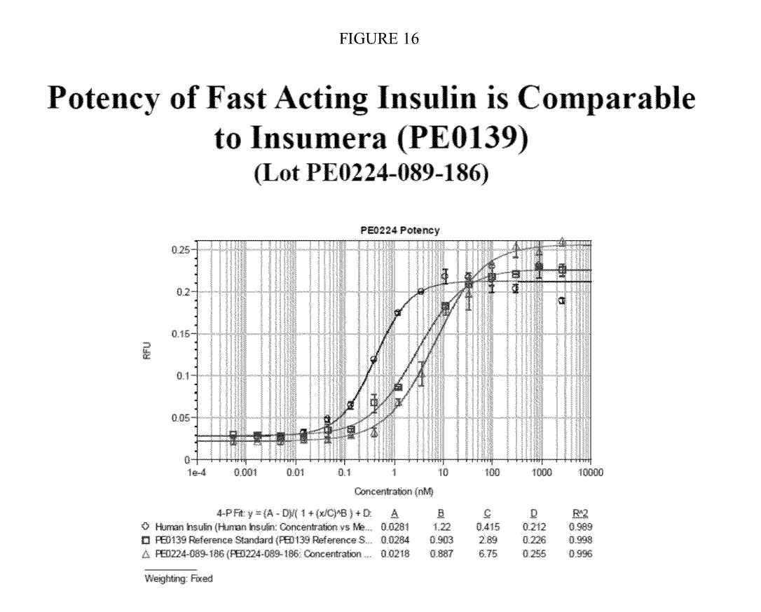

Studies were also conducted to evaluate the pharmacokinetics (PK) of INSUMERA treatment. In diabetic swine, either a single s.c. injection (FIG. 13A) or daily s.c. injections for 2 weeks (FIG. 13B) regimen was followed. The results show that INSUMERA achieves a long half-life with a small peak to trough ratio following a subcutaneous injection.

### Insulin/Receptor Interactions

A goal of the following studies is to perform additional tests of insulin peptide and insulin peptide/protein constructs binding to human insulin and IGF-1 receptors.

Analysis conditions: binding studies were performed at 25° C. using a Biacore S51 optical biosensor equipped with a nickel-charged NTA sensor chip and equilibrated with running buffer (10 mM HEPES, 150 mM NaCl, 0.01% Tween-20, pH 7.8). For these studies, a molecular mass of 5808 Da was assumed for the LCR-1054 insulin peptide and 50,000 Da for INS-ELP(B30) and PE0139.

A screen of INS-ELP(B30) and PE0139 at higher concentration was also performed. In earlier tests, receptor binding of INS-ELP(B30) and PE0139 was not observed. These two analytes were re-tested at 3 μM and included LCR-1054 (at 600 nM) as a positive control.

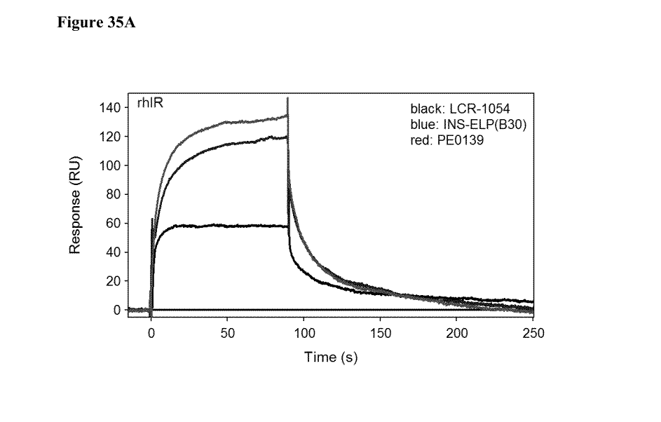

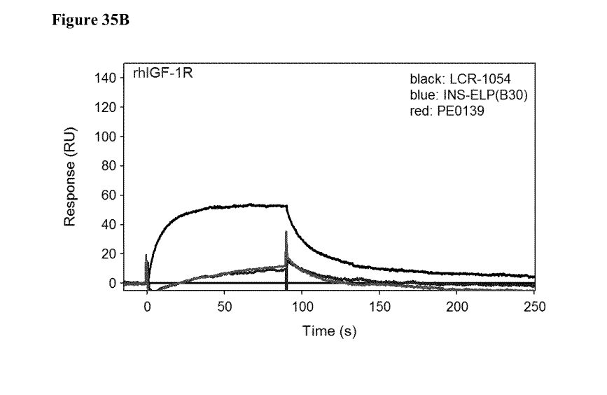

At 3 μM, INS-ELP(B30) and PE0139 showed binding to the insulin receptor surface (FIG. 35A). At 3 μM, INS-ELP(B30) and PE0139 did not display significant binding to the IGF-1 receptor (FIG. 35B).

To investigate these interactions in more detail, fresh surfaces of rhIR and rhIGF-1R were prepared. The His-tagged rhIR was captured to a density of about 1200 RU. IGF-1R was amine-coupled on the same sensor chip to a density of 7500 RU. The activity of these new rhIR and rhIGF-1R surfaces was confirmed by the binding of 600 nM LCR-1054.

Concentration Series of INS-ELP(B30) and PE0139.

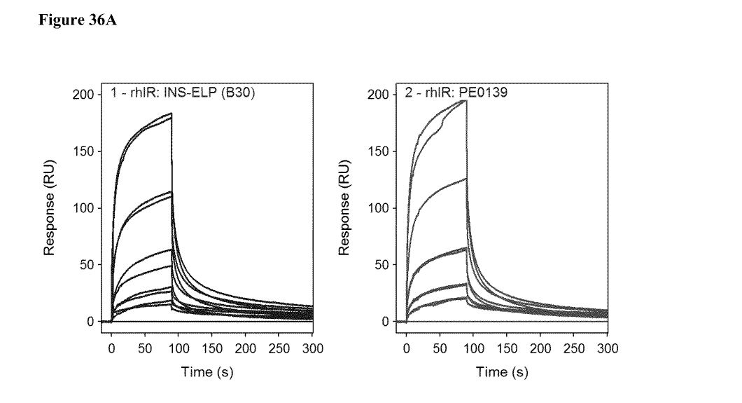

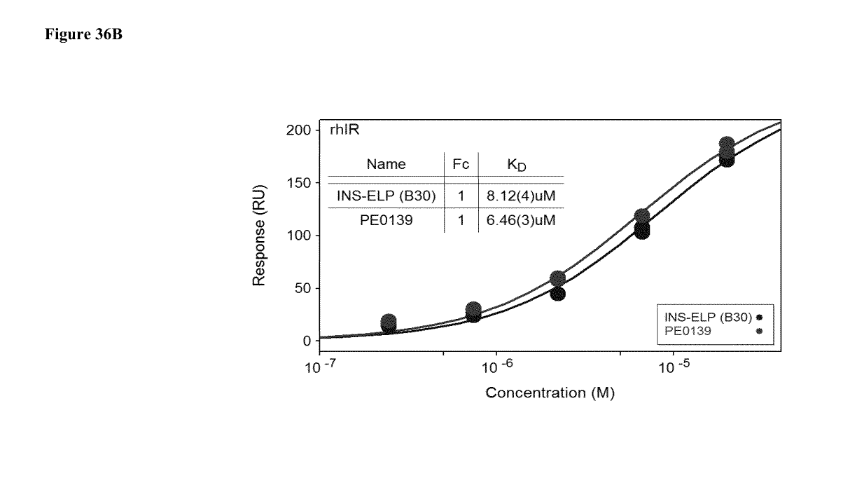

INS-ELP(B30) and PE0139 were tested in a three-fold dilution series starting at 20 μM for binding to the two receptor surfaces. The two analytes showed similar binding to the insulin receptor (FIG. 36A)

The responses were plotted against analyte concentration and fit to a simple binding isotherm to obtain affinity estimates of these interactions (FIG. 36B).

In contrast, neither analyte showed reliable binding to the rhIGF-1R surface. In fact, the responses from this surface are negative at the higher concentrations because the analytes showed more binding to the reference surface (a region of the sensor chip that had no protein attached) than to the immobilized IGF-1R.

Accordingly, the present example shows that, inter alia, INS-ELP(B30) and PE0139 bind to insulin receptor with affinities of about 6-8 uM under these analysis conditions and that INS-ELP(B30) and PE0139 do not show any detectable binding to IGF-1 receptor.

Binding of the constructs to the IGF-1R was subsequently investigated using a cell-based assay for receptor activation, using cells engineered to express IGF receptor. Using this system, the rapid-acting and sustained-release constructs did exhibit some activation of the IGF receptor, but at a level significantly less than insulin (data not shown).

### Example 2

### Rapid Acting Insulin Constructs

To create “fast acting” insulin ELP fusions, the proinsulin sequence was genetically fused to ELP polymers of shorter length than the 120 ELP pentamer repeats in pPE0139. These shorter polymers have transition temperatures well above physiological conditions, effectively removing the sustained release functionality of the polymer but retain the ability to prevent insulin multimerization and confer stability in solution.

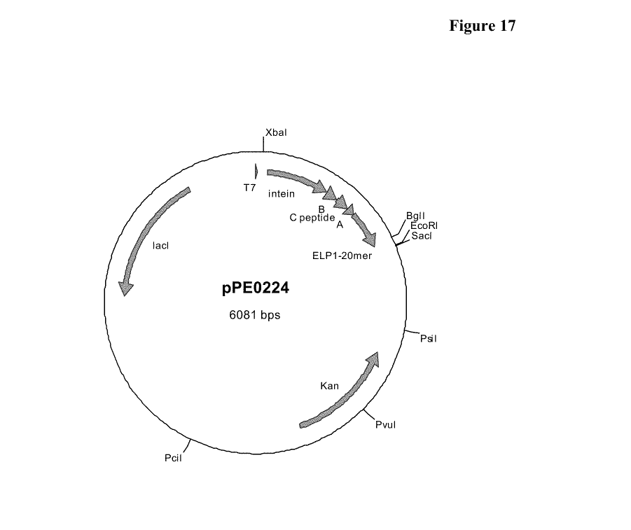

The proinsulin nucleotide sequence was synthesized and sub-cloned using restriction enzymes into pET based vector pPE0221. This positions the proinsulin at the N-terminus of the ELP1-20 sequence to make plasmid pPE0224 (FIG. 17).

FIG. 18 shows the amino acid sequence of a proinsulin ELP1-20 fusion protein (SEQ ID NO:16). The proinsulin sequence (underlined) is fused to the ELP1-20 sequence (bold).

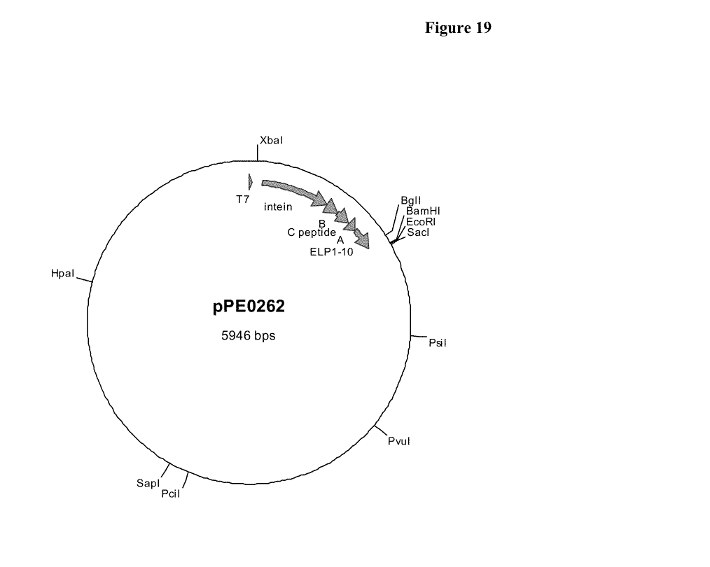

To build a proinsulin fused to a polymer of shorter length than the ELP1-20 the proinsulin nucleotide sequence was synthesized and sub-cloned into pET based vector pPE0215. This positions the proinsulin at the N-terminus of the ELP1-10 sequence to make plasmid pPE0262 (FIG. 19).

FIG. 20 shows the amino acid sequence of a proinsulin ELP1-10 fusion protein (SEQ ID NO:17). The proinsulin sequence (underlined) is fused to the ELP1-10 sequence (bold).

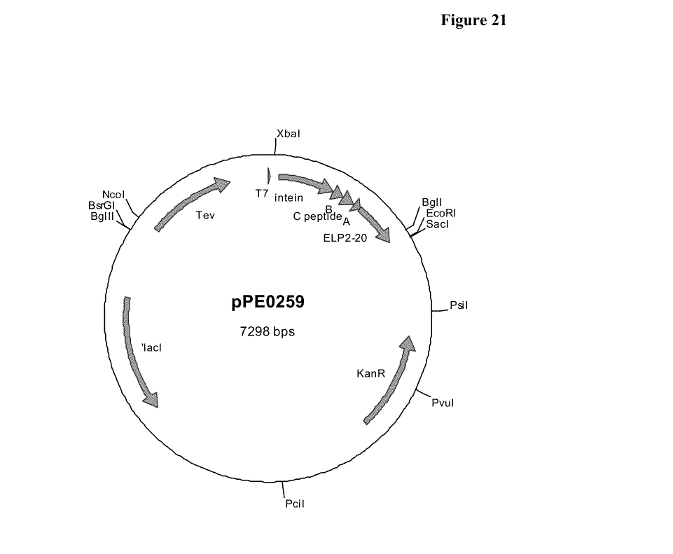

To build a proinsulin fused to a polymer of even higher transition temperature than the ELP1-20, the proinsulin nucleotide sequence was synthesized and sub-cloned into pET based vector pPE0245. This positions the proinsulin at the N-terminus of the ELP2-20 sequence to make plasmid pPE0259 (FIG. 21). The ELP2-20 polymer uses alanine and glycine as the VPGXG (SEQ ID NO: 3) guest residue in a 1:1 ratio decreasing the hydrophobicity of the polymer compared with the ELP1-20.

FIG. 22 shows the amino acid sequence of a proinsulin ELP2-20 fusion protein (SEQ ID NO:18). The proinsulin sequence (underlined) is fused to the ELP2-20 sequence (bold).

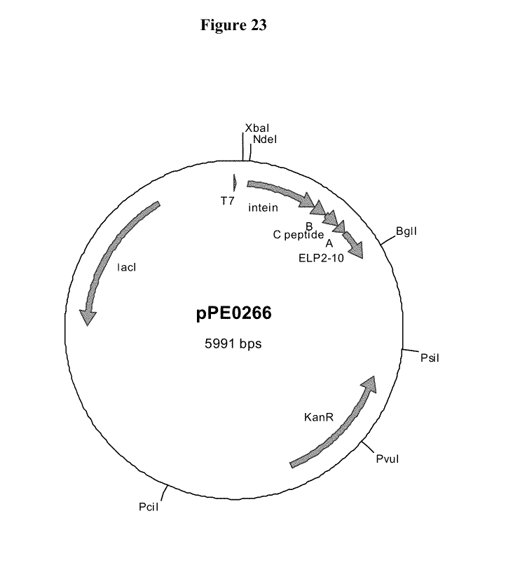

To build a proinsulin fused to a polymer of shorter length than the ELP2-20 the proinsulin nucleotide sequence was synthesized and sub-cloned into pET based vector pPE0265. This positions the proinsulin at the N-terminus of the ELP2-10 sequence to make plasmid pPE0266 (FIG. 23).

FIG. 24 shows the amino acid sequence of a proinsulin ELP2-10 fusion protein (SEQ ID NO:19). The proinsulin sequence (underlined) is fused to the ELP2-10 sequence (bold).

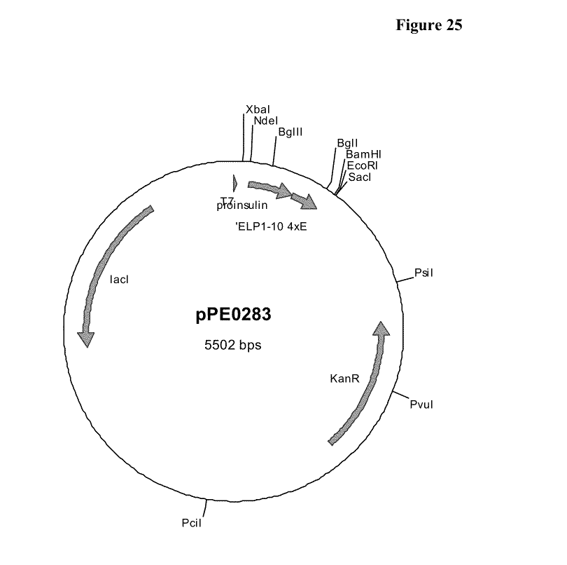

To build a proinsulin fused to a polymer of a greater negative charge than the ELP1-10 the proinsulin nucleotide sequence was synthesized and sub-cloned into pET based vector pPE0274. This positions the proinsulin at the N-terminus of the ELP1-10 sequence with 4 glutamic acid residues inserted between the ELP pentamers to make plasmid pPE0283 (FIG. 25). The inserted glutamic acid residues increases the negative charge of the polymer.

FIG. 26 shows the amino acid sequence of a proinsulin ELP1-10 (4xGlu) fusion protein (SEQ ID NO:20). The proinsulin sequence (underlined) is fused to the ELP1-10 (4xGlu) sequence (bold) with the inserted glutamic acid residues noted (bold underlined).

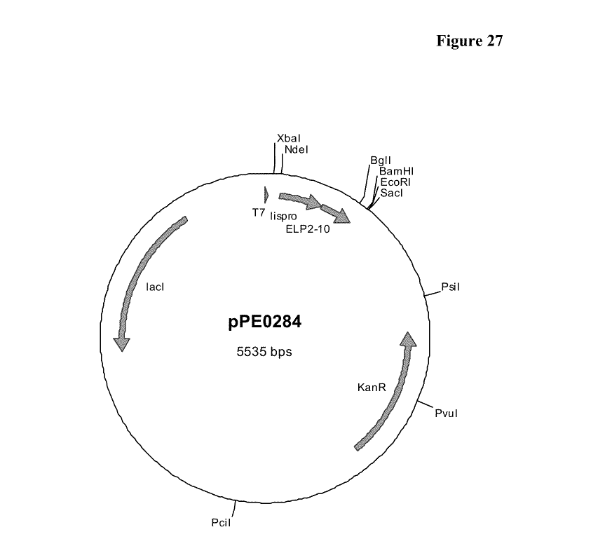

To build a lispro insulin fused to an ELP polymer the lispro nucleotide sequence was synthesized and sub-cloned into pET based vector pPE0265. This positions the lispro insulin at the N-terminus of the ELP1-10 sequence to make plasmid pPE0284 (FIG. 27).

FIG. 28 shows the amino acid sequence of a lispro ELP1-10 fusion protein (SEQ ID NO: 21). The lispro sequence (underlined) is fused to the ELP1-10 sequence (bold).

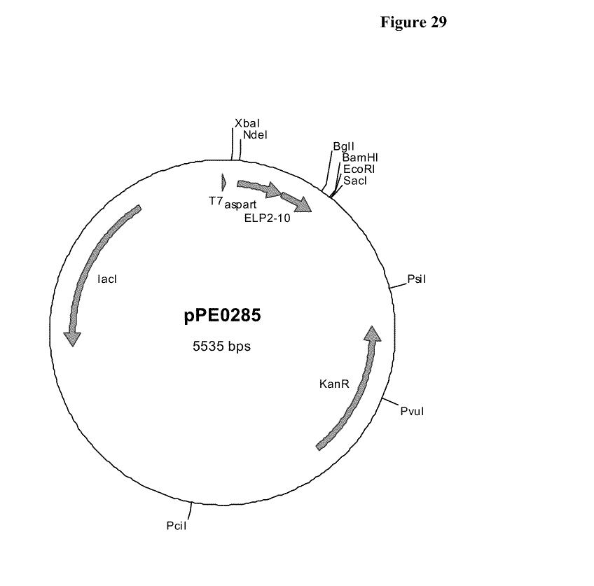

To build an insulin aspart fused to an ELP polymer the aspart nucleotide sequence was synthesized and sub-cloned into pET based vector pPE0265. This positions the insulin aspart at the N-terminus of the ELP1-10 sequence to make plasmid pPE0285 (FIG. 29).

FIG. 30 shows the amino acid sequence of an aspart ELP1-10 fusion protein (SEQ ID NO:22). The aspart sequence (underlined) is fused to the ELP1-10 sequence (bold).

### Fermentation

Proinsulin ELP fusions described above were expressed in the intracellular fraction of E. coli under control of the T7 promoter in a fed-batch fermentation process. The glycerol cell stock was expanded using a two-stage shake flask seed train in semi-defined, animal-free medium (ECPM+Proline) with glycerol as the primary carbon source and yeast extract as the primary nitrogen source. After sufficient cell density was achieved in the seed train, the culture was transferred to a fermentor containing the same medium as the seed train. Process parameters (pH, temperature, dissolved oxygen) were maintained at set point via PID control. The culture grew until it reached stationary phase whereupon a glycerol/yeast extract/magnesium sulfate feed was initiated. The culture was maintained under carbon limitation and induction of the promoter was achieved using IPTG. At the end of the fermentation, the culture was centrifuged to separate the biomass containing the Insulin ELP fusion from the spent medium. The cell paste was stored at −70° C. until subsequent purification.

### Initial Purification

Frozen cell paste was resuspended in lysis buffer containing 2M Urea (for dissociation of Insulin ELP) and mixed until homogenous. Lysis was achieved using a microfluidizer to disrupt the cell membranes. An initial clarification of the lysate was performed by centrifugation. A two stage tangential flow filtration (TFF) system was used to further clarify and concentrate the product. The Proinsulin ELP fusion was passed over a HIC column as a capture step and host cell contaminants were washed away. The product was eluted using a gradient to fractionate any product-related impurities (degraded species). TFF buffer exchange using a pH 7.0-9.0 Tris buffer was performed on the selected fractions to remove residual salt prior to subsequent conversion from Proinsulin ELP to Insulin ELP.

### Enzymatic Processing of Proinsulin ELP Fusion

The captured Proinsulin ELP was converted to Insulin ELP through enzymatic digestion of the C-peptide using recombinant Trypsin and Carboxypeptidase B, at a ratio of 0.05 ug/mg Proinsulin and 2.0 ug/mg Proinsulin ELP, respectively. The reaction was incubated at room temperature (20-25° C.) for 3 to 4 hours until the C-peptide was fully cleaved yield mature Insulin ELP.

### Final Purification

Following conversion to Insulin ELP, the residual enzymes and product variants were removed using two approaches. One, the residual enzymes were removed by reprocessing the Insulin ELP over the capture HIC column where the Insulin ELP bound to the column and the residual enzymes passed through. Final polishing steps following enzyme removal included a Cation Exchange column to remove product variants, host cell protein, endotoxin and DNA and an Anion Exchange column to remove any residual DNA and endotoxin. Alternatively, removal of the enzymes and the polishing step can be achieved through the Cation Exchange column simultaneously. The enzymes tightly bind to the resin allowing for selective elution of Insulin ELP to separate the product from the enzymes, host cell protein, endotoxin and DNA. A subsequent Anion Exchange column removed any remaining contaminants. Products were subsequently concentrated and diafiltered into formulation buffer (e.g. 20 mM histidine, 110 mM NaCl).

### Effect of PE0244 (Fast-Acting Insulin) and PE0139 (Sustained Release Insulin) on Glucose Levels in a Rodent Model of Type 1 Diabetes.

Diabetes was induced in mice by the administration of streptozotocin (70 mg/kg IP) by established methods. Twelve days later, mice were fasted for 4 hr and then administered PE0244, PE0139, a co-formulation of PE0244 and PE0139, or vehicle. Blood glucose measurements were taken for up to 4 hr post-injection. Fig Y shows that PE0244 caused a rapid reduction in blood glucose due to the rapid entry of insulin into the circulation, but the effect began to wear off towards the end of the observation period. In contrast, PE0139 had a significantly slower onset of action due to slow release from the depot at the injection site, but the effect was sustained through the observation period. The combination formulation showed a rapid onset, plus a sustained reduction in blood glucose, clearly showing that the two molecules retained their properties in this co-formulation.

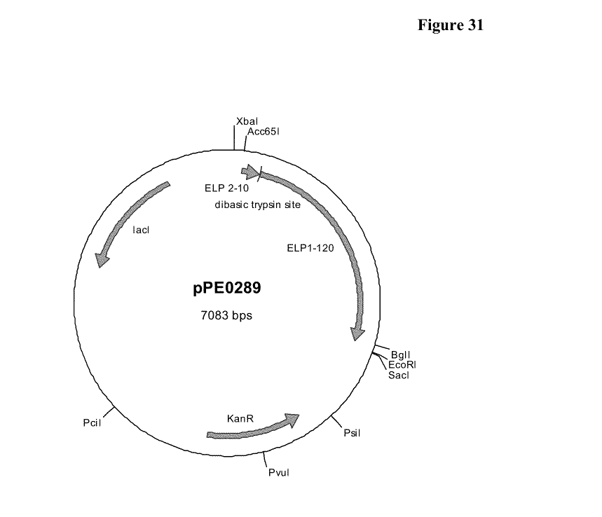

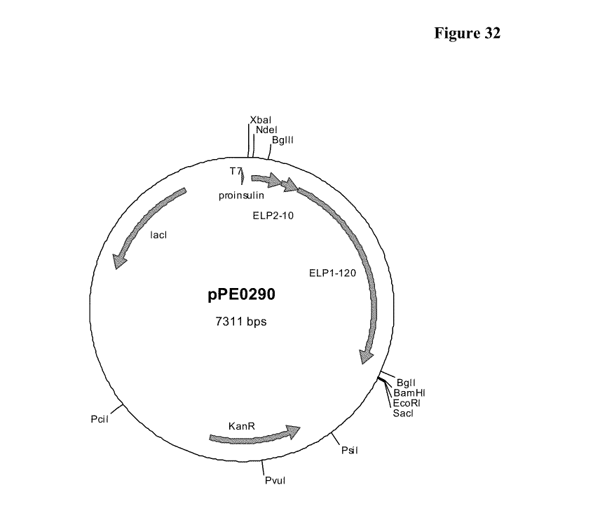

The ELP1-120 sequence improves the solubility in the E. coli cytoplasm as well as the downstream processing efficiency of insulin fusions. To take advantage of this with the shorter ELP polymer fusions, the proinsulin ELP2-10 polymer was fused to the N-terminus of the ELP1-120mer polymer with a trypsin cleavage site between the two. This allows the polymers to be expressed together and separated during the enzymatic step of the purification process resulting in a proinsulin ELP2-10 fusion like the one expressed in pPE0266. To build this “breakaway” construct, the nucleotide sequence of the ELP2-10 polymer along with a double arginine tryptic site was subcloned into plasmid pPB1031 to create plasmid pPE0289 (FIG. 31). This positions the ELP2-10 polymer on the N-terminus of the ELP1-120 polymer. Next the proinsulin nucleotide sequence was cloned onto the N-terminus of the ELP2-10 sequence to make plasmid pPE0290 (FIG. 32).

FIG. 33 shows the amino acid sequence of a proinsulin ELP2-10 breakway protein (SEQ ID NO:23). The proinsulin sequence (underlined) is fused to the ELP1-10 sequence (bold) which in turn is fused to the ELP1-120 sequence (italics). The tryptic site between the ELPS in noted (bold underline).

### Processing of a Breakaway Construct

The ELP1-120 sequence improves the solubility in the E. coli cytoplasm as well as the downstream processing efficiency of insulin fusions. To take advantage of this with the shorter ELP polymer fusions, the proinsulin ELP2-10 polymer was fused to the N-terminus of the ELP1-120mer polymer with a trypsin cleavage site between the two. This allows the polymers to be expressed together and separated during the enzymatic step of the purification process resulting in a proinsulin ELP2-10 fusion like the one expressed in pPE0266. To build this “breakaway” construct, the nucleotide sequence of the ELP2-10 polymer along with a double arginine tryptic site was subcloned into plasmid pPB1031 to create plasmid pPE0289 (FIG. 31). This positions the ELP2-10 polymer on the N-terminus of the ELP1-120 polymer. Next the proinsulin nucleotide sequence was cloned onto the N-terminus of the ELP2-10 sequence to make plasmid pPE0290 (FIG. 32).

FIG. 33 shows the amino acid sequence of a proinsulin ELP2-10 breakway protein (SEQ ID NO:23). The proinsulin sequence (underlined) is fused to the ELP1-10 sequence (bold) which in turn is fused to the ELP1-120 sequence (italics). The tryptic site between the ELPS in noted (bold underline).

### Example 3

### Combination Therapy with Sustained Release Insulin and GLP-1 Receptor Agonist

Combination therapy with the sustained release insulin (PE0139) and a GLP-1 Receptor agonist was evaluated. The GLP-1 receptor agonist is represented by SEQ ID NO:32 (PB1023), which includes an ELP1-120 fusion, where X is Val, Gly and Ala at a ratio of about 5:3:2. The following results were obtained with db/db mice, and the effects on fasting glucose and post-prandial glucose at different doses of the active agents were evaluated. Mice were faster for about 3.5 hours prior to time 0, with IPGTT at about 240 minutes.

FIG. 37 shows that dose escalation of PE0139 in db/db mice exhibits increasing effect on fasting and post-prandial glucose.

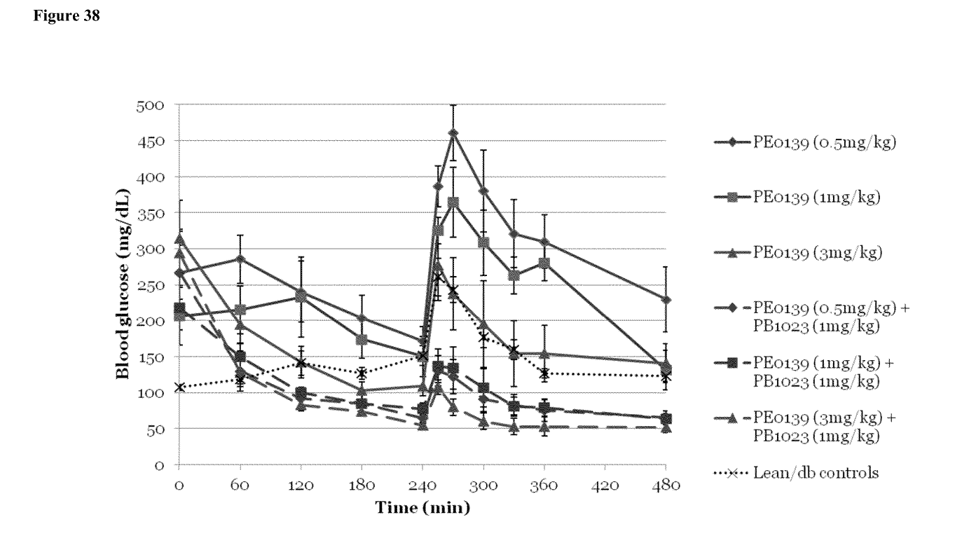

FIG. 38 shows that the addition of a fixed dose of PB1023 (GLP-1-ELP1-120) to PE0139 further enhances post-prandial control in db/db mice.

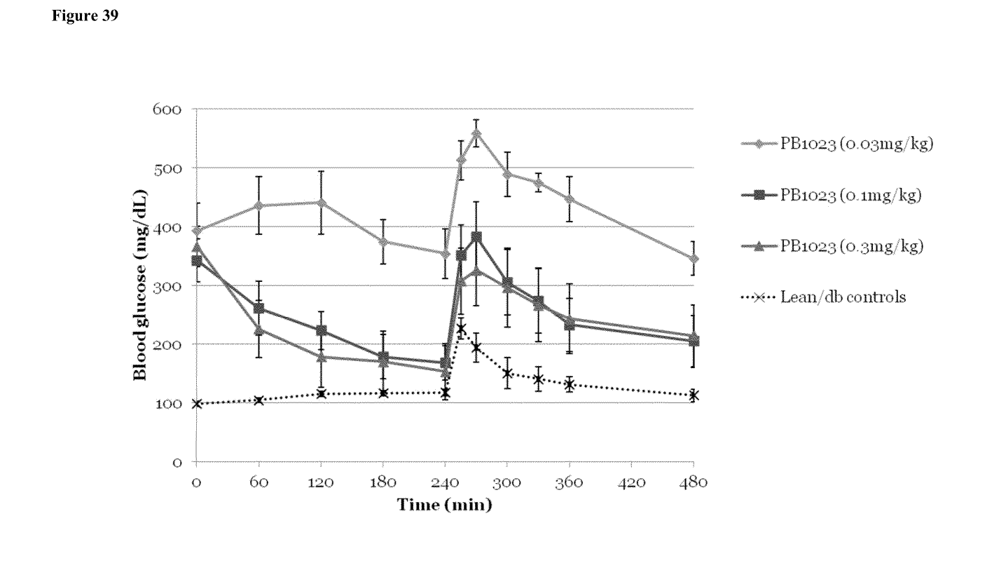

FIG. 39 shows that PB1023 dose escalation shows increasing effect on fasting and post-prandial glucose in db/db mice.

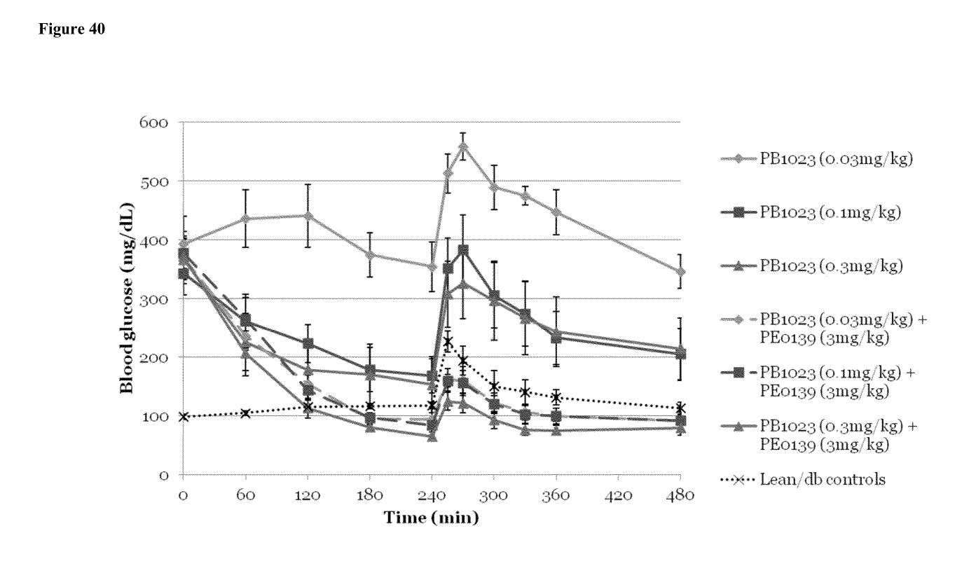

FIG. 40 shows that the addition of fixed dose of PE0139 to PB1023 enhances fasting and post-prandial control in db/db mice.

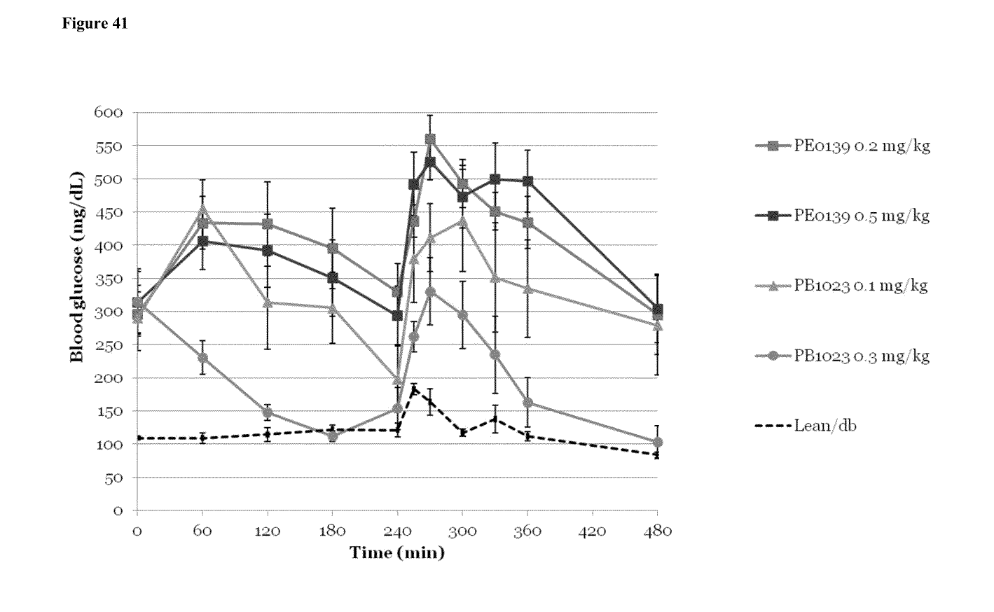

FIG. 41 shows that PE0139 dose escalation shows little effect on blood glucose, and that PB1023 dose escalation shows increasing effect on fasting glucose, and limited impact on post-prandial glucose.

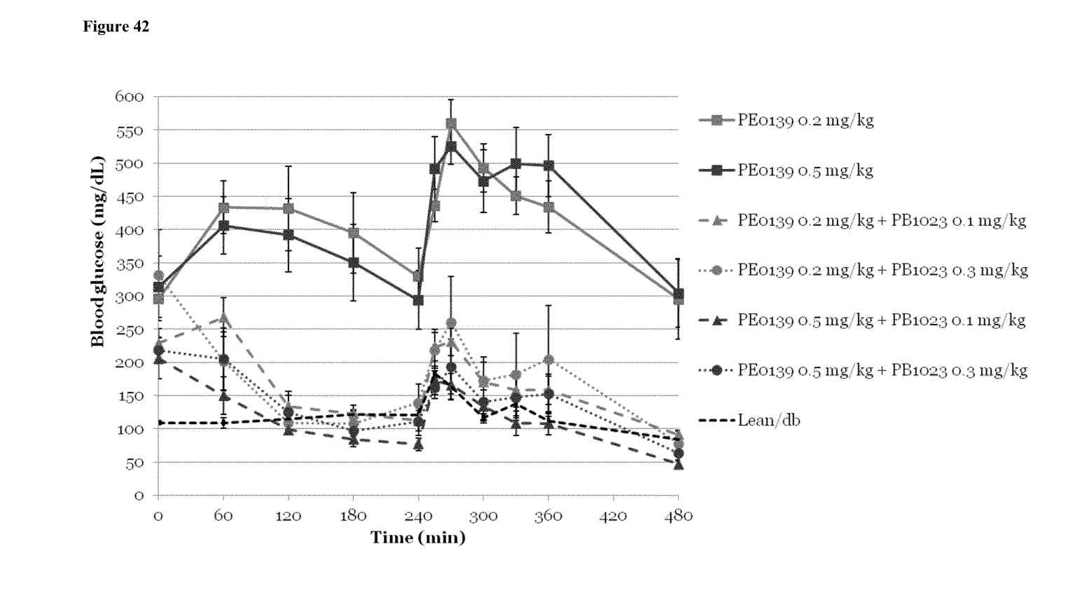

FIG. 42 shows that combination of low doses of PE0139 and PB1023 have enhanced fasting and post-prandial control.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A therapeutic protein comprising an insulin B chain and insulin A chain, and a fusion partner of the insulin A chain of from 5 to 200 amino acids that inhibits insulin multimer formation.

2. The therapeutic protein of claim 1, wherein the therapeutic protein exhibits reduced activation of the IGF receptor as compared to native insulin.

3. The therapeutic protein of claim 1 or claim 2, wherein the fusion partner has from about 50 to about 150 amino acids.

4. The therapeutic protein of any one of claims 1 to 3, wherein the fusion partner has an extended conformation comprising repeating beta-turns.

5. The therapeutic protein of any one of claims 1 to 4, wherein the sequence of the fusion partner contains less than about 30% of hydrophobic residues selected from leucine, isoleucine, valine, methionine, cysteine, histidine, phenylalanine, tyrosine, and tryptophan.

6. The therapeutic protein of claim 5, wherein the therapeutic protein does not exhibit a phase transition at body temperature.

7. The therapeutic protein of claim 5 or 6, formulated in a pharmaceutically compatible solution for subcutaneous injection.

8. The therapeutic protein of claim 7, wherein the therapeutic protein reaches peak insulin action within 45 minutes of injection.

9. The therapeutic protein of any one of claims 1 to 8, wherein the A chain and B chain have the amino acid sequence of SEQ ID NO:13, optionally having from 1 to 8 modifications independently selected from a amino acid insertion, amino acid deletion, or amino acid substitution.

10. The therapeutic protein of claim 9, wherein one or more of positions 3, 28, 29, and 30 of the insulin B chain are substituted, and/or position 21 of the A chain is substituted.

11. The therapeutic protein of any one of claims 1 to 10, wherein the A chain and B chain are bound by one or more disulfide bonds, or are attached through a peptide or chemical linker.

12. The therapeutic protein of any one of claims 1 to 11, wherein the fusion partner comprises ELP units.

13. The therapeutic protein of claim 12, wherein the fusion partner comprises from about 10 to about 25 repeats of VPGXG (SEQ ID NO: 3), wherein X is independently selected from Val, Gly, and Ala, or comprises from about 10 to 25 repeats of AVGVP (SEQ ID NO:4).

14. The therapeutic protein of any one of claims 1 to 13, wherein the fusion partner contains from 1 to about 10 negatively charged amino acids.

15. The therapeutic protein of claim 13 or 14, where G and A are present in an approximately equivalent amount as residue X, and V is present as X at a frequency of more than G and/or A.

16. The therapeutic protein of any one of claims 1 to 15, wherein the fusion protein has the sequence of SEQ ID NO:16 or SEQ ID NO:17.

17. The therapeutic protein of claim 12, wherein the fusion partner comprises from about 10 to about 25 VPGXG units (SEQ ID NO: 3), where X is independently selected from V, G, and A, where G and A are present in an approximately equivalent amount as residue X, and V is present as X at a frequency of less than G or A.

18. The therapeutic protein of claim 17, wherein the fusion protein has the sequence of SEQ ID NO:18, or SEQ ID NO:19, SEQ ID NO:21, or SEQ ID NO:22.

19. The therapeutic protein of claim 17, where from 2 to 6 positively charged residues.

20. The therapeutic protein of claim 19, wherein the fusion protein has the amino acid sequence of SEQ ID NO:20.

21. A pharmaceutical composition comprising a first fusion protein of any one of claims 1 to 20, and a second fusion protein comprising an insulin B chain and insulin A chain, and a fusion partner of from about 400 to about 1000 amino acids, the second fusion protein exhibiting a phase transition at body temperature so as to provide a sustained release of the second fusion protein from the injection site.

22. The pharmaceutical composition of claim 21, wherein the second fusion protein has a fusion partner with an extended conformation comprising repeating beta-turns.

23. The pharmaceutical composition of claim 22, wherein the first fusion protein has a fusion partner comprising [VPGXG]₁₂₀ (SEQ ID NO:29), where each X is selected from V, G, and A, and wherein the ratio of V:G:A is about 5:3:2.

24. The pharmaceutical composition of any one of claims 21 to 23, for once daily administration.

25. The pharmaceutical composition of any one of claims 21 to 24, wherein the first and second fusion proteins are separated from a single fusion protein by proteolytic processing.

26. The pharmaceutical composition of any one of claims 21 to 25, wherein the pharmaceutical composition contains about a 1:1 ratio of the first and second fusion proteins.

27. A method for treating diabetes or hypoinsulinemea, comprising: administering the therapeutic agent of any one of claims 1 to 20, or the pharmaceutical composition of claims 21 to 26 to a patient in need thereof.

28. The method of claim 27, wherein the patient has type 1 or type 2 diabetes or is prediabetic.

29. The method of claim 27, wherein the therapeutic protein is administered from 1 to 3 times daily, or the pharmaceutical composition is administered once daily.

30. The method of any one of claims 26 to 29, wherein the therapeutic protein or pharmaceutical composition is administered about 15 minutes or less before a meal.

31. A method for treating diabetes or hypoinsulinemea, comprising: administering a rapid-acting prandial insulin, and a long-acting basal insulin to a patient, wherein the rapid-acting insulin is the therapeutic protein of any one of claims 1 to 20; and/or the long-acting insulin comprises an insulin B chain and insulin A chain, and a fusion partner of from about 400 to about 1000 amino acids, the long-acting insulin exhibiting a phase transition at body temperature so as to provide a sustained release from the injection site.

32. The method of claim 31, wherein the long-acting insulin has a fusion partner with an extended conformation comprising repeating beta-turns.

33. The method of claim 31 or 32, wherein the long-acting insulin is administered once weekly or once daily.

34. The method of any one of claims 31 to 33, wherein the rapid acting insulin is administered from once to three times daily prior to commencing a meal.

35. The method of any one of claims 31 to 34, wherein the patient has type 1 or type 2 diabetes or is prediabetic.

36. The method of any one of claims 31 to 35, wherein the rapid-acting insulin and the long-acting insulin are administered by a pump system that monitors blood glucose.

37. A method for treating diabetes, metabolic disease, or clinical obesity, comprising: administering a regimen of a long-acting insulin comprising an insulin B chain and insulin A chain, and a fusion partner of from about 400 to about 1000 amino acids, the long-acting insulin exhibiting a phase transition at body temperature so as to provide a sustained release from the injection site, and administering a regimen of a GLP-1 receptor agonist.

38. The method of claim 37, wherein the GLP-1 receptor agonist is GLP1-ELP1-120.

39. The method of claim 37 or 38, wherein the long-acting insulin and the GLP-1 receptor agonist each have a fusion partner comprising [VPGXG]₁₂₀ (SEQ ID NO:29), where each X is selected from V, G, and A, and wherein the ratio of V:G:A is about 5:3:2.

40. The method of any one of claims 37 to 39, wherein the insulin and the GLP-1 receptor agonist are formulated separately or together.

41. The method of any one of claims 37 to 40, wherein the insulin and the GLP-1 receptor agonist are co-formulated and administered about once weekly.

42. A fusion protein comprising a rapid-acting insulin and a long-acting insulin, and a protease site connecting the rapid acting insulin and the long-acting insulin.
